(19) United States
(12) Patent Application Publication
(10) Pub. No.: US 2024/0301032 A1
(43) Pub. Date: Sep. 12, 2024

(54) SUBSTRATES COMPRISING ELASTIN-LIKE POLYPEPTIDES AND CALCIUM IONS

(71) Applicant: MINTECH-V, LLC, WILMINGTON, DE (US)

(72) Inventors: Abshar Hasan, Nottingham (GB); Sherif Ahmed Abdelsalam Elsharkawy, London (GB); Alvaro Mata Chavarria, Nottingham (GB)

(73) Assignee: MINTECH-V, LLC, WILMINGTON, DE (US)

(21) Appl. No.: 18/434,379

(22) Filed: Feb. 6, 2024

Related U.S. Application Data
(60) Provisional application No. 63/483,475, filed on Feb. 6, 2023.

(51) Int. Cl. C07K 14/78; C30B 7/04

(52) U.S. Cl. CPC C07K 14/78; C30B 7/04

(57) ABSTRACT

The disclosure is directed towards polypeptide substrates and methods of synthesis thereof. Such substrates can be embedded with calcium ions from a number of ionic sources. These calcium-embedded, polypeptide substrates can be used to grow a variety of crystal structures including flower-shaped, onion-shaped, and needle-like crystal structures. As such, the disclosure is additionally directed towards methods of crystal growth from polypeptide substrates. Compositions of the disclosure can be used in a wide variety of medical and other applications.

### PRIORITY AND INCORPORATION BY REFERENCE

This application claims priority to U.S. Provisional Application Ser. No. 63/483,475, filed on Feb. 6, 2023, the entire disclosure of which is hereby incorporated by reference herein for any and all purposes. This application hereby incorporates by reference the entire disclosure of U.S. patent application Ser. No. 17/588,579, filed on Jan. 31, 2022, including the sequence listings by reference herein for any and all purposes.

### SEQUENCE LISTING

The application contains a Sequence Listing which has been submitted electronically in .XML format and is hereby incorporated by reference in its entirety. Said .XML copy, created on May 16, 2024, is named “067608-0002US1” and is 51 kilobytes in size. The sequence listing contained in this .XML file is part of the specification and is hereby incorporated by reference herein in its entirety.

### TECHNICAL FIELD OF INVENTION

The present invention relates to polypeptide substrates incorporating calcium ions and processes for their formation. The present invention also relates to crystals produced from these substrates and processes for their production.

### BACKGROUND

Elastin-like polypeptides (ELPs) are a type of protein-like molecule that consist of repeating pentapeptide sequences of Val-Pro-Gly-Xaa-Gly (VPGXG), where X is any amino acid apart from proline. These molecules undergo a phase transition at a certain transition temperature (Tₜ), which results in the transition from a soluble to an insoluble form. In solutions with a temperature lower than Tₜ, free polymer chains remain in an unordered state showing full hydration (the soluble form). In solutions with temperatures exceeding Tt, polymer chains show a more ordered structure (known as the β-spiral), stabilized by hydrophobic interactions and intramolecular type β structures increasing the association of polymer chains.

However, ELP membranes are limited in their ability to access more varied crystal morphologies and form crystal structures inside the membrane.

Therefore, there exists a need to address limitations of membranes existing in the art.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

The present disclosure relates to a polypeptide substrate which can form crystal structures on both the interior and exterior of the membrane. Such substrates can be embedded with calcium ions.

The present disclosure relates to a polypeptide substrate which can form more varied crystal structures including flower-shaped, onion-shaped, and needle-like crystals.

The present disclosure relates to providing crystals having improved stiffness, toughness, hardness, wear resistance, compressive strength, and acid resistance.

The present disclosure relates to a process of growing the mineralized structures epitaxially from underlying crystal structures. Underlying crystal structures include bone tissue and surrounding dental enamel. This is facilitated by the incorporation of calcium ions into polypeptide substrates.

The present disclosure relates to a process for forming a crystal structure from a polypeptide substrate incorporating calcium ions.

The present disclosure relates to increased formation of amyloid-like ensembles from polypeptide molecules using Ca ions.

The present disclosure relates to incorporating polypeptide substrates with or without crystal structures into a variety of devices and applications.

The present disclosure relates to methods and compositions for overcoming or mitigating at least one problem of the prior art, whether expressly disclosed herein or not.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIGS. 1A-1E display onion-shaped crystal structures according to aspects of the present disclosure.

FIGS. 2A-2H display different stages of onion-shaped crystal growth according to aspects of the present disclosure.

FIGS. 3A-3B display topography of perpendicular spiky crystal structures at varied calcium ion concentrations.

FIGS. 4A-4G display flower-shaped crystal structures according to aspects of the present disclosure.

FIGS. 5A-5D display crystal fusion inside polypeptide membranes according to the present disclosure.

FIGS. 6A-6D display mineralization (e.g., crystallization) occurring within thicker membrane cross-sections according to the present disclosure.

FIGS. 7A-7B displays a use of compositions disclosed herein to regenerate bone tissue according to the present disclosure.

FIGS. 8A-8D display characterizations of re-mineralized underlying crystal structures according to the present disclosure.

FIGS. 9A-9B display mineralization on nylon (FIG. 9A) and titanium (FIG. 9B) scaffolds according to the present disclosure.

FIGS. 10A-10D display underlying mechanisms in pathologies compared to mineralizing systems disclosed herein.

FIGS. 11A-11I display positive effects of cells growing on membranes in vitro according to the present disclosure.

FIGS. 12A-12F display characteristics of cells growing on membranes in vitro according to the present disclosure.

FIGS. 13A-13L display positive effects of cells growing on membranes in vitro according to the present disclosure.

FIGS. 14A-14D display characteristics of cells growing on membranes in vitro according to the present disclosure.

FIGS. 15A-15C display images of in vivo bone regeneration performed on rabbits according to the present disclosure.

FIGS. 16A-16D display characteristics of in vivo bone regeneration performed on rabbits according to the present disclosure.

FIGS. 17A-17F display images of in vivo bone regeneration performed on rabbits according to the present disclosure.

FIGS. 18A-18D display characteristics of in vivo bone regeneration performed on rabbits according to the present disclosure.

FIGS. 19A-19E display polarized microscopy of crosslinked elastin (FIGS. 19A and 19B). SEM images of membranes before mineralization of elastin (FIG. 19C). Spherulites are revealed by SEM formed on elastin membrane (FIGS. 19D and 19E) for 8 days.

FIGS. 20A-20F display SEM images of natural elastin (FIGS. 20A and 20B) and natural collagen (FIGS. 20C and 20D) in bovine heart aorta showing elastin entangled long filaments and collagen fibrils with visible trile helix unit, respectively. TEM images of aorta showing both elastin and collagen organization (FIG. 20E) across the tissue, which in higher magnification collagen fibrils are showing the gap-zone (FIG. 20F).

FIGS. 21A-21DD display histological analysis of aorta and mitral valve before and after enzymatic digestion.

FIGS. 22A-22J display tissue samples from aorta according to the present disclosure.

FIGS. 23A-23M display structural analysis of mineralization on aorta and mitral valve tissues after 8 days mineralization

### DETAILED DESCRIPTION OF THE INVENTION

The present disclosure can be understood more readily by reference to the following detailed description, examples, drawings, and claims, and their previous and following description. However, before the present compositions, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

### I. Definitions

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present disclosure. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used herein, “amino acids” are organic compounds that contains both amino and carboxylic acid functional groups and serve as the building blocks for polypeptide or protein molecules.

As used herein, a “polypeptide” is a linear organic polymer consisting of a large number of amino-acid residues bonded together in a chain, forming part of (or the whole of) a protein molecule.

Other terms known in the art relating to the present disclosure exist and are to be understood as used in the art, unless otherwise specified.

### II. Polypeptide Substrates

The present disclosure relates to polypeptide substrates. Such substrates further comprise calcium ions, wherein calcium ions are embedded within the substrates. Additional ions and particles are also useful when embedded in substrates including but not limited to hydroxyapatite nanoparticles, fluoride ions, zinc oxide ions, and others. Substrates include a variety of structures to be used in numerous applications, disclosed herein. Substrates include but are not limited to membranes, particles, coatings, slurries, hydrogels, and the like which are conducive to crystal growth. Particles include but are not limited to nanoparticles and microparticles. Crystal growth is discussed herein. Crystals can grow both from the inside and on the outside, or a combination thereof, of substrates described herein.

The present disclosure relates to polypeptides used in substrates. Polypeptides include but are not limited to intrinsically disordered polypeptides and elastin-like polypeptides (ELPs). Intrinsically disordered polypeptides include but are not limited to elastin, resilin, amelogenin, ameloblastin, and the like. Other exemplary polypeptides include but are not limited to collagen, enamelin. Additional exemplary polypeptides include but are not limited to any polypeptide found in the enamel matrix. Though substrates disclosed herein may be referred to as ELP substrates, it is understood that the aforementioned additional polypeptides are capable of being used, as well. As used herein, ELP may comprise elastin-like recombinamers (ELRs).

The present disclosure relates to embedded calcium ions within polypeptide substrates. Embedding calcium ions alters the secondary structure of the substrates and creates nucleation points inside the substrates. The creation of nucleation points allows for specifically tailoring crystal growth morphology and function, as described herein. Additional exemplary secondary structure alterations include but are not limited to changes in secondary structures, amyloid and amyloid-like formations, changes in mechanical characteristics including stiffness, altered crosslinking, changes in swelling capacity, and the like. (Elsharkawy, S., et al., Nature Communications, 9(2145): 1-12 (2018)). Beneficially, this opens up the possibility for formation of crystals both inside and outside the substrate.

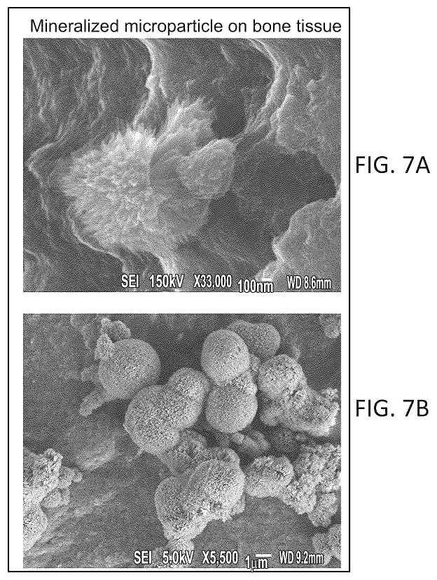

Additionally, the incorporation of calcium ions within the polypeptide substrate allows the formation of novel crystal structures, together with crystal structures possessing improved mechanical properties and improved integration with bone and underlying crystal structures including but not limited to dental enamel. Underlying crystal structures refers to any pre-existing crystal structure with which crystals can be integrated with using methods and embodiments disclosed herein. Moreover, surprisingly, the inventors discovered that crystal structures formed from polypeptide substrate comprising calcium ions embedded within the substrate have similar mechanical characteristics including stiffness to natural dental enamel. FIGS. 7A-7B displays use of compositions of the present disclosure for bone regeneration.

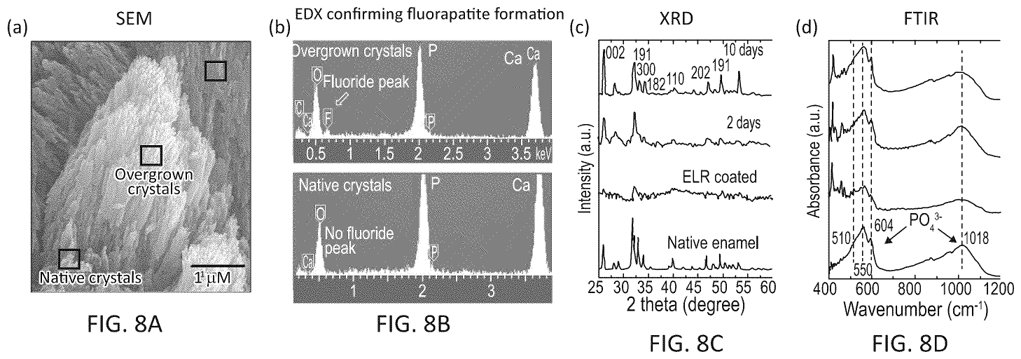

FIGS. 8A-8D further display physical characterization of integration of compositions disclosed herein with underlying crystal structures. FIG. 8A shows an SEM micrograph of re-mineralized enamel prism. FIG. 8B shows EDX analysis confirmed formation of fluoride substituted apatite (fluorapatite, FAp) nanocrystals crystals on top of native hydroxyapatite crystals. FIG. 8C shows XRD characterization showing formation of fluorapatite crystalline phase with the formation of typical phosphate peaks at 604 and 565 cm⁻¹ (v₄(PO₄)) and 1035 cm⁻¹ v₃(PO₄), indicating apatite formation.

As a non-limiting example, ELP molecules are known to form amyloid-like ensembles with moderate levels of β conformation including β-sheets and β-turns. Moderate levels include levels of about 40% β conformation. The novel addition of embedded calcium ions in polypeptide substrates enhances the formation of amyloid-like ensembles. β conformation has been shown to be increased up to about 80%.

In a number of applications, amyloid-like ensembles are highly desired. As a non-limiting example, amyloid-like ensembles are desired for biomineralization of underlying crystal structures including but not limited to bone structures and dental enamel.

The percentage of ELP in the substrate may be at least 1%, 2%, 3%, 4% or at least 5% by weight of the polypeptide substrate. In some embodiments, the percentage by weight of ELP in the substrate may be no more than 20%, 15%, 10%, or 8% by weight.

The percentage by weight of ELP in the substrate may be from 1-20% by weight, 1-15% by weight, 1-10% by weight. Most preferably, 5% by weight.

As a non-limiting example where calcium ions are embedded in a polypeptide substrate, calcium ions may be Ca²⁺.

The calcium ions may be provided by any substance capable of donating calcium ions known in the art. As non-limiting examples, calcium-ion-donating substances include but are not limited to CaCl₂·nH₂O (where n=0, 1, 2, 4, and 6), calcium carbonate (CaCO₃), calcium phosphates (e.g., hydroxyapatite, octacalcium phosphate), calcium nitrate, any calcium-based mineral, and the like. One of skill in the art would be aware of additional, suitable substances capable of donating Ca²⁺ ions.

Preferably, the calcium ions are provided by compounds selected from the group consisting of CaCl₂).

The present disclosure relates to optimized calcium ion concentrations by weight of the substrate. Overly elevated calcium ion concentrations lead to increased mineralization on the surface of the substrate. This prevents diffusion of calcium, phosphorus, and fluorine ions into the substrate, thus reducing the number of crystal structures forming inside the bulk of the substrate.

The calcium ions may be present in an amount of at least 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.12%, 0.14%,0.16%0.18%, 0.20%, 0.22%0.24%, 0.28%, or 0.30% by weight of the substrate. In some embodiments, the calcium ions may be present in an amount of no more than 1.5%, 1.4%, 1.3%, 1.2%, 1.1%, 1%, 0.9%, 0.8%, 0.7%, 0.6%, or 0.5% by weight of the substrate.

Preferably the calcium ions may be present in an amount of 0.005-1.5%, 0.005-0.7% by weight of the substrate, more preferably 0.001-0.5% by weight of the substrate.

Surprisingly, it has been observed that the number of crystal structures formed decreases with an increase in the amount of calcium ions present by weight in the substrate. Advantageously, this means that the inventors have found an polypeptide substrate which can be used to tune the morphology or organisation of any hierarchical crystal structure formed from the substrate.

The ELP may include various sequences, such as those referenced in U.S. application Ser. No. 17/588,579, which is incorporated by reference herein for any and all purposes.

As an example, the ELP includes a peptide sequence selected from the group consisting of

(a)
MGSSHHHHHHSSGLVPRGSHMESLLP-
[VPGIGVPGIGVPGKGVPGIGVPGIGVPGIGVPGIGVPGKGVPGIGVPG
IGAVTGRGDSPASSVPGIGVPGIGVPGKGVPGIGVPGIGVPGIGVPGIG
VPGKGVPGIGVPGIG]6-V
(b)
MESLLP-
VPGIGVPGIGVPGKGVPGIGVPGIGEEIQIGHIPREDVDYHLYPVPGIG
VPGIGVPGKGVPGIGVPGIGVGVAPGVGVAPGVGVAPG]10-V
(c)
MESLLP-VPGIG VPGIG VPGKG VPGIG VPGIG VPGIG VPG
GI VPGKG VPGIG VPGIG]12-V
(d)
MESLLP-[((VPGIG)2VPGKG(VPGIG)2)2-DDDEEKFLRRIGR
F(G-VPGIG)2VPGKG(VPGIG)2)2]3-V
(e)
MESLLP-[((VPGIG)2VPGKG(VPGIG)2)2-DDDEEKFLRRIGR
F(G-(VPGIG)2VPGKG(VPGIG)2)2]3-(VPAVG)20-V
(f)
MESLLP-[((VPGIG)2VPGKG(VPGIG)2)2-DDDEEKFLRRIGR
FG-((VPGIG)2VPGKG(VPGIG)2)2]3-(VPAVG)20-
[((VPGIG)2VPGKG(VPGIG)2)2-DDDEEKFLRRIGRFG-
((VPGIG)2VPGKG(VPGIG)2)2]3-V
and
(g)
MESLLP-(VPGVG VPGVG VPGEG VPGVG VPGVG)10-
(VPAVG)40-V.

By way of non-limiting example, the ELP may be or comprise a statherin-ELP. As another example, the ELP may be or comprise i) a pentapeptide sequence selected from the group consisting of Gly-X-X-X-X, X-Gly-X-X-X, X-X-Gly-X-X, X-X-X-Gly-X and X-X-X-X-Gly, (GXXXX, XGXXX, XXGXX, XXXGX, XXXXG), wherein X is any amino acid apart from proline, (ii) RGDS-ELP, and/or (iii) Statherin-ELP. As a further example, the ELP may consist of or comprise the sequence MGSSHHHHHHSSGLVPRGSHMESLLP-[((VPGIG)2VPGKG(VPGIG)2)2-DDDEEKFLRRIGRFG-((VPGIG)2VPGKG(VPGIG)2)2]3-V.

The ELP may comprise a pentapeptide selected from the group consisting of Gly-X-X-X-X, X-Gly-X-X-X, X-X-Gly-X-X, X-X-X-Gly-X and X-X-X-X-Gly, (GXXXX, XGXXX, XXGXX, XXXGX, XXXXG), wherein X is any amino acid apart from proline. Preferably, X is an amino acid selected from the group consisting of V, P, G, S, F and I.

The ELP may comprise the tropoelastin recurrent motif Val-Pro-Gly-X-Gly (VPGXG), where X is any amino acid apart from proline.

The ELP may comprises the tropoelastin recurrent motif Pro-Gly-Ile-Pro-Gly (PGIPG).

The ELP may comprise the tropoelastin recurrent motif Pro-Val-Gly-Ser-Gly (PVGSG).

The ELP may comprise the tropoelastin recurrent motif Val-Gly-Phe-Pro-Gly (VGFPG).

Native elastin itself may also be used with recurrent motif Val-Pro-Gly-Val-Gly.

ELPs are recombinant proteins. They can be produced in bacterial cells or purchased.

An exemplary polypeptide substrate disclosed herein includes polypeptide membranes including ELP membranes. Membranes disclosed herein may range from about 1 micrometre to about 2000 micrometres in thickness. In a preferred embodiment, membranes may range from about 40 micrometre up to about 80 micrometres in thickness.

In one embodiment, the ELP membrane may have a thickness of from 0.5 mm-1.5 mm, 0.6 mm-1.4 mm, 0.7-1.3 mm, 0.8 mm-1.2 mm, or 0.9-1.1 mm.

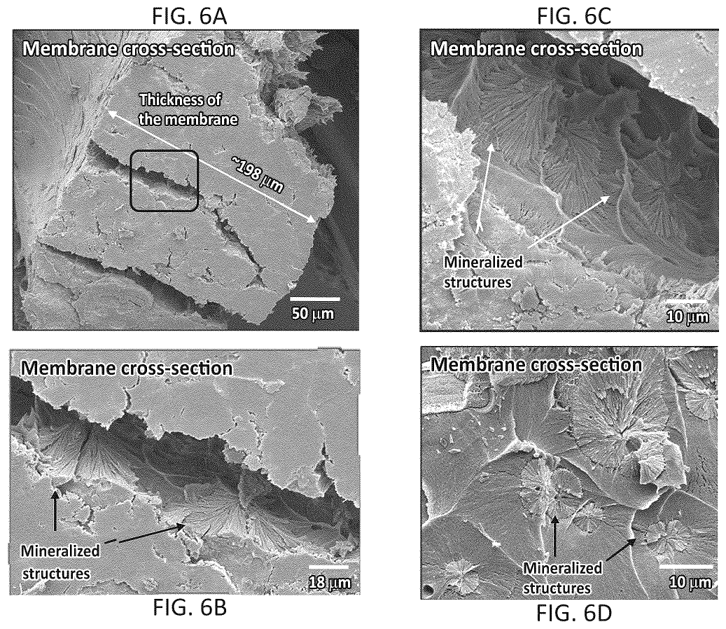

By incorporating calcium ions inside the membrane, the inventors surprisingly discovered that thicker membranes were able to form crystal structures, as shown in FIGS. 6A-6D. Moreover, the formed crystals possessed increased thickness when compared with existing crystal structures produced from existing membranes not comprising calcium ions.

The ELP membrane may be cross-linked by a cross-linker. Cross-linkers bind polypeptide molecules both intramolecularly and intermolecularly. This function assists in the formation of stable amyloid-like polypeptide ensembles.

A cross-linker is an inorganic or organic reagent that reacts with either a carboxylic group or an amine group of a polypeptide substrate through covalent bonds, or non-covalent bonds such as electrostatic, hydrogen bonds, or Van der Waals. The polypeptide substrate may be cross-linked by chemical cross-linking, enzymatic cross-linking by tissue transglutaminase, photoinitiated and/or y-irradiation cross-linking.

Preferably, cross-linker is hexamethyl diisocyanate. Additional cross-linkers include but are not limited to glutaraldehyde, sodium tripolyphosphate, Riboflavin, phosphated riboflavin, 4 arm polyethylene glycol (PEG), succinimidyl glutarate, and PEG (Succinimidyl Carboxymethyl Ester)2.

The polypeptide substrate may further comprise collagen, amelogenin, bone sialoprotein, enamelin or phosphorylated serine. The polypeptide substrate may comprise graphene, carbon nanotubules, and/or quantum dots. The polypeptide substrate may comprise sugar, proteins, inorganic particles and/or peptides. The skilled person would also understand that a wide range of solvent soluble materials can be incorporated into the polypeptide substrate.

The polypeptide substrate may be biocompatible. By “biocompatible” it is meant that the substrate is not harmful or toxic to living tissue.

The polypeptide substrate may have a β-spiral conformation. The presence of a β-spiral conformation can be confirmed using circular dichroism (CD) and Fourier transform infrared (FTIR) spectroscopy.

### III. Methods of Polypeptide Substrate Synthesis

The present disclosure relates to a method of synthesis for forming polypeptide substrates. Various types of substrates may be synthesized including membranes, coatings, particles, hydrogels, and the like. Particles include but are not limited to nanoparticles and microparticles. Substrates described herein can be formed with an elastin-like polypeptide (ELP) or any polypeptide disclosed herein. ELP solutions described below can be prepared with any polypeptide disclosed herein in order to form various polypeptide substrates.

As a non-limiting example, the present disclosure relates to forming ELP membranes, the process comprising the steps of:
a) mixing an elastin-like polypeptide with a source of calcium ions and a solvent to form an ELP solution; and
b) applying the solution onto a surface to form a membrane.

Step b) may comprise drop casting the solution onto a surface.

The ELP may be present in an amount of at least 1%, 2%, 3%, 4% or at least 5% by volume of the ELP solution. In one embodiment, the ELP may be present in an amount of no more than 20%, 15%, 10%, or 8% of the ELP solution.

The ELP may be present in an amount of from 1-20%, 1-15%, or 1-10% by volume of the ELP solution. Most preferably, the ELP is present in an amount of 15% by volume of the ELP solution.

The source of calcium ions may be any substance capable of donating calcium ions known in the art. Exemplary calcium sources are described herein. The skilled person would be aware of additional, suitable substances capable of donating calcium ions.

Preferably, the calcium ions are Ca²⁺.

The calcium ions may be provided by any substance capable of donating calcium ions known in the art. As non-limiting examples, calcium-ion-donating substances include but are not limited to CaCl₂) nH₂O (where n=0, 1, 2, 4, and 6), calcium carbonate (CaCO₃), calcium phosphates (e.g., hydroxyapatite, octacalcium phosphate), calcium nitrate, any calcium-based mineral, and the like. One of skill in the art would be aware of additional, suitable substances capable of donating Ca²⁺ ions.

The source of calcium ions may be present in an amount of at least 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.12%, 0.14%,0.16%0.18%, 0.20%, 0.22%0.24%, 0.28%, or 0.30% by volume of the ELP solution. In one embodiment, the source of calcium ions may be present in an amount of no more than 1.5%, 1.4%, 1.3%, 1.2%, 1.1%, 1%, 0.9%, 0.8%, 0.7%, 0.6%, or 0.5% of the ELP solution by volume.

Preferably the source of calcium ions may be present in an amount of 0.005-1.5% by volume or 0.005-0.7% by volume of the ELP solution, more preferably 0.001-0.5% by volume of the ELP solution.

ELP may be present in 5% w/v of the ELP solution. Additional, non-limiting examples include ELP present in about 0.1% w/v up to about 20% w/v, about 1% w/v up to about 19% w/v, about 2% w/v up to about 18% w/v, about 3% w/v up to about 17% w/v, about 4% w/v up to about 16% w/v, about 5% w/v up to about 15% w/v, about 6% w/v up to about 14% w/v, about 7% w/v up to about 13% w/v, about 8% w/v up to about 12% w/v, and about 9% w/v up to about 11% w/v of the ELP solution.

The solvent may be any solvent suitable for dissolving ELP. Such solvents would be well known to the person skilled in the art. Non-limiting examples of solvents include but are not limited to 100% water, ethanol mixtures, ethanol-water mixtures, dimethylformamide (DMF), dimethyl sulfoxide (DMSO), and the like. Additional exemplary solvents include phosphate buffer saline (PBS) solution. Such a solvent has been shown to be able to be used with beta-[tris(hydroxymethyl)phosphino]propionic acid (THPP) as a crosslinker.

Preferably, the solvent is dimethylformamide (DMF) and/or dimethyl sulfoxide (DMSO).

The step b) of applying the solution onto a surface may comprise the steps of dropping the solution onto the surface and evaporating the solvents.

The surface may be any surface suitable for receiving the ELP solution. Such surfaces would be well known to the skilled person.

Preferably, the surface is a polymeric material.

Preferably, the surface is poly(dimethylsiloxane) (PDMS). Any surface known in the art may be used, however.

The step a) may further comprise the step of mixing the ELP solution with a cross linker, which cross-linker may be in solution.

Preferably, the cross-linker is hexamethyl diisocyanate. Additional exemplary crosslinkers are disclosed herein.

As a non-limiting example, one synthetic process for creating an ELP membrane includes a first step of dissolving ELP molecules into a mixture of anhydrous dimethyformamide (DMF) and dimethyl sulfoxide (DMSO) solvents at a 9:1 ratio. Such solution is then mixed with CaCl₂·2H₂O at a range of concentrations from about 0.01% up to about 0.5% w/v at room temperature inside a humidity controlled (e.g., <20% humidity) glovebox. The ELP-Ca solution can then be crosslinked with hexamethylene diisocyanate (HDI) or glutaraldehyde at about 0.5% up to about 5% v/v before being drop-casted on top of a polydimethylsiloxane (PDMS) surface and dried overnight.

### IV. Crystal Formations

The present disclosure relates to crystal formations growing in combination with polypeptide substrates described herein. Crystal formations include but are not limited to nanocrystals. Crystal formations can grow in a variety of ways with relation to a polypeptide substrate. As a non-limiting example, crystals can grow where the nucleation only takes place within the substrate. The incorporation of ions, including calcium ions, into the polypeptide substrate, also allows for crystals to be grown by nucleation both inside and outside the substrate. As a non-limiting example epitaxial growth of crystals at an underlying crystal-ELP coating interface occurred by growing crystals inside the polypeptide substrate and towards the substrate surface. This can lead to remineralization. Underlying crystals include but are not limited to bone tissue and dental enamel.

The crystal may be apatite. ELP-mediated apatite nanocrystals exhibit similar physical and chemical properties as that of enamel and bone crystals. Apatite crystals additionally grow epitaxially on underlying crystal structures including but not limited to bone tissue and dental enamel and bone to recreate their native microstructure.

Apatite refers to a phosphate mineral. Apatites are flexible structures with wide range of optional substitutions that can happen in their lattice at both cation and anion positions. Apatites have the general formula A₁₀(BOn)₆X₂ (alternatively As(BOn)₃X). A may be a divalent cation selected from the group consisting of Ca²⁺, Sr²⁺, Ba²⁺ and Pb²⁺. BOn is an anionic complex, such as an anionic complex selected from the group consisting of PO₄⁻, AsO₄³⁻, VO₄³⁻ or CO₃²⁻. X is generally an anion. Preferably, X is selected from the group consisting of OH, F and CI.

Apatites have hexagonal crystallographic symmetry. Such geometry may additionally be described as hexagonal flat ended geometry. The space group of apatites is usually (P63/m) where the 6-fold c-axis is perpendicular to 3 a-axes at 120° to one another with some lower symmetry analogues.

The apatite may be selected from the group comprising fluroapatite, hydroxyapatite and chlorapatite.

Preferably, the apatite is fluorapatite. Fluroapatite is a phosphate mineral with the general formula Ca₅(PO₄)₃F. Fluroapatite is alternatively referred to as Ca₁₀(PO₄)F₂ or FAp.

The apatite may be hydroxyapatite. Hydroxyapatite is a phosphate mineral with the general formula Ca₅(PO₄)₃(OH).

The crystal may be located at least partly inside the bulk of the polypeptide substrate. The crystals may be located at least partly outside the bulk of the polypeptide substrate.

The crystal may be located on the polypeptide substrate surface.

The crystal may be located partly inside the bulk of the polypeptide substrate and partly outside the bulk of the polypeptide substrate.

The crystal may be located partly inside the bulk of the polypeptide substrate and partly on the surface of the polypeptide substrate.

The crystal may have a hierarchical structure.

By “hierarchical structure” it is meant a structure having different structures at different length scales. Hierarchy is a structural feature observed in natural tissues including but not limited to enamel, nacre, and bone. Hierarchy is difficult to achieve synthetically.

The present disclosure relates to growing structures with different shapes inside the bulk of a substrate in a tailored fashion. This allows for the formation of differently organised hierarchically mineralized structures. Specifically tailoring hierarchy allows fusing of crystals, as described herein. This facilitates “controlled fusions” where a bundle of nanocrystals fuses into a single large crystal. Tailored hierarchy allows for control and limiting capabilities concerning the extent of fusion.

The crystal may comprise nanostructures, microstructures and macrostructures assembled in a hierarchal order across multiple length-scales. The length-scales can be crystallographic, nanometre, micrometre, one hundred micrometre and millimetre. Preferably, each level of hierarchy comprises morphologically distinct structures.

At the crystallographic length-scale, the material may be apatite.

In growth from underlying crystal structures, hexagonal apatite nanocrystals were observed to grow epitaxially from the underlying crystal structure-ELP interface outwards through the ELP matrix. Underlying crystal structures include but are not limited to bone tissue and dental enamel. In this case, the growth is regulated and limited by the thickness of the ELP matrix, generating apatite layer similar to the thickness of the ELP coating.

At the nanometre length scale the structures of the invention may comprise nanocrystals.

Crystals disclosed herein may be antimicrobial in nature. The term ‘antimicrobial’ generally refers to substances or components that can kill, or inhibit the growth of, microorganisms. As a non-limiting example, spiky nanocrystals described further below grow perpendicular to a substrate and disrupt bacterial membranes when bacteria come in contact to the substrate, thus exhibiting antibacterial properties.

### A. Onion-Like Nanocrystal Formations

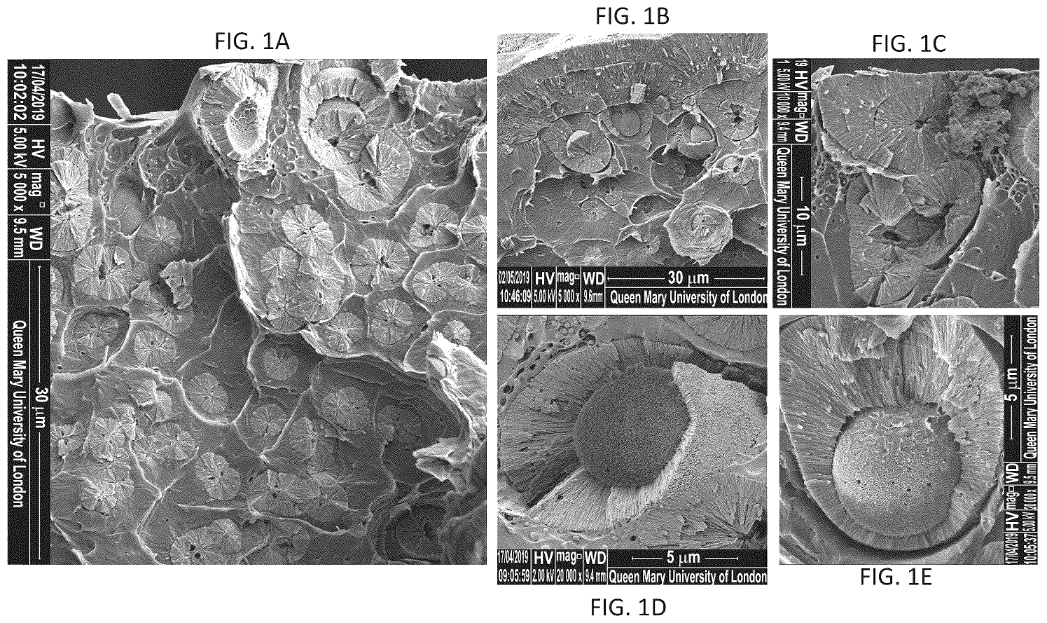

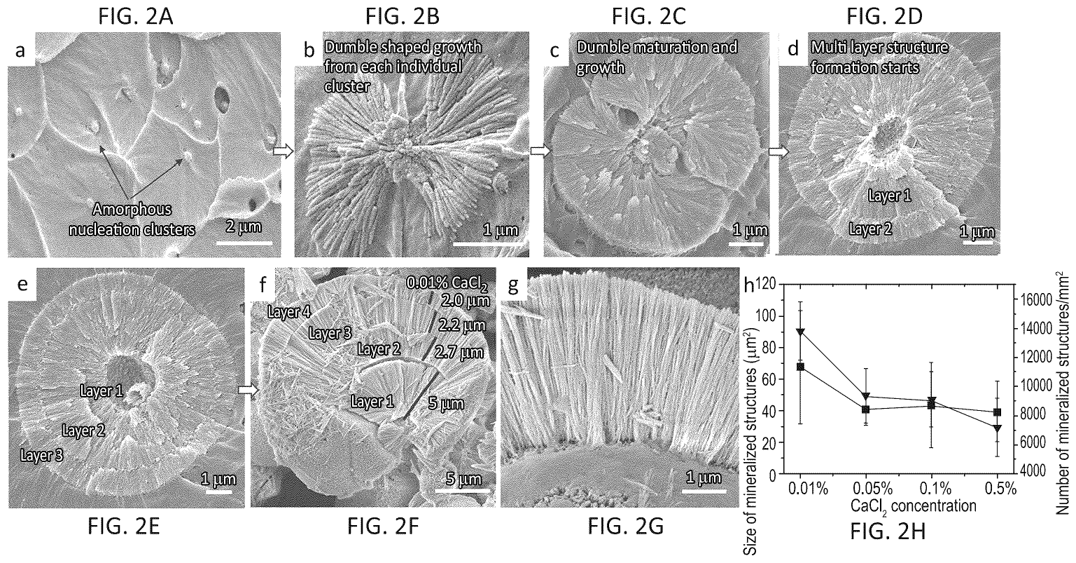

The nanocrystals may be arranged in “onion-like”, formations in that layers of crystals are superposed, as shown in FIGS. 1A-1E. Onion-like growth patterns include hexagonal nanocrystals nucleating, growing, and organizing into dumbbell-shaped structures to form the inner most layer of the onion. This is followed by deposition of multiple layers of nanocrystals on top of each other. Stages of onion-shaped growth patterns are shown in FIGS. 2A-2H. Onion-like growth differs from previous methods of crystal growth including concentric ring growth where apatite nanocrystals nucleate within the bulk of an ELP matrix to form the root of a mineralized structure. Nanocrystals from this concentric ring root grow, emerge, and spread radially on the surface of the substrate. When the aligned nanocrystals emerged out of the bulk and onto the substrate surface, they organized into microscopic circular concentric rings, different from onion-like growth patterns.

Each adjacent layer of nanocrystals may be separated by a layer of ELP. Various layers of ELP and nanocrystals can be formed by replenishing mineralizing solution during crystal formation. As a non-limiting example, mineralizing solution can be replenished every 2 days, as it was observed that the pH of the solution falls from 6 to 4 every 2 days. Replenishing the mineralizing solution results in nucleation and growth of new layers of crystals on top of previous layers. This process is described further below.

The number of layers of nanocrystals may decrease as the percentage by weight of calcium ions incorporated into a polypeptide substrate increases.

The layers of onion-like nanocrystals may be located inside the bulk of the substrate.

The onions structures are visible on micro-scale. At nanoscale they are composed of hexagonal apatite nanocrystals.

The onion-like shaped nanocrystals comprise an exemplary diameter of at least 10 nm, 11 nm, 12 nm, 13 nm, 14 nm, 15 nm, 16 nm, 17 nm, 18 nm, 19 nm, and 20 nm. Diameters of onion-like nanocrystals are determined by the length of the nanocrystals growing axially from the substrate surface in onion-like “layers.” Diameters can be increased by subjecting onion-like shaped nanocrystals to mineralization solutions for longer periods, as disclosed herein. The use of thicker substrates for growing onion-like shaped nanocrystals can also increase the diameter.

The onion-like nanocrystals may create a mineralized layer extending from the substrate surface with a thickness of at least 1 μm, 5 μm, 10 μm, 15 μm, 20 μm, 25 μm and no more than 100 μm, 90 μm, 80 μm, 70 μm, 60 μm. The thickness may be from 1-100 μm, 10-90 μm, 20-80 μm, 30-70 μm, 40-60 μm. The overall mineralized layer may be composed of multiple individual layers of onion-like nanocrystals. As a non-limiting example, a single layer of onion-like nanocrystals may range from about 1 μm up to about 5 μm. By growing multiple such layers on top of each other, thicknesses disclosed above are achieved.

### B. Needle-Like or Spiky Nanocrystal Formations

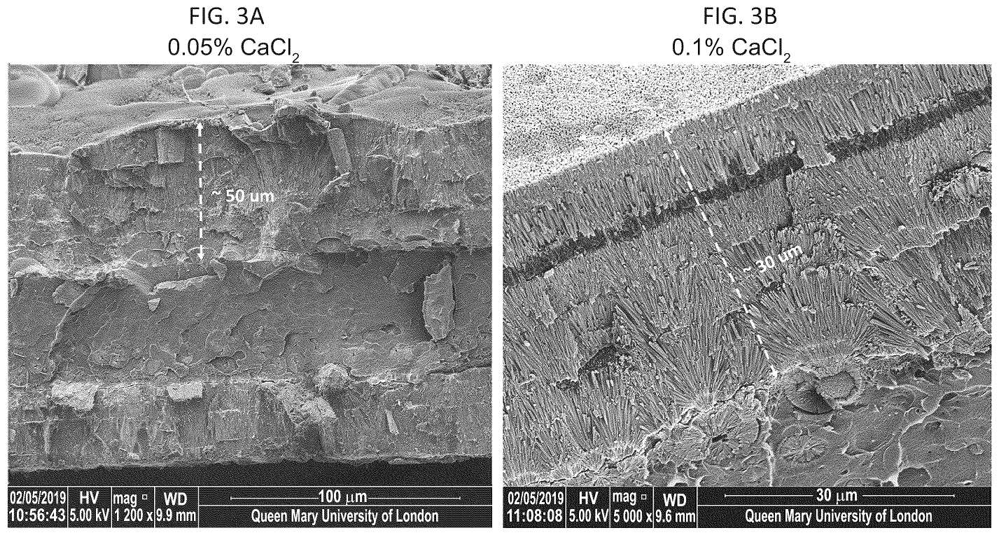

Crystal formations may comprise one or more needle shaped or “spiky” nanocrystals, as shown in FIGS. 3A-3B. The crystals may comprise a plurality of needle shaped nanocrystals. Needle shaped or “spiky” nanocrystals grow to form the thick mineralized layer on top of a substrate. The needle shaped nanocrystals may be located on the polypeptide substrate surface.

The needle shaped nanocrystals may be orientated perpendicular to the substrate surface.

In one embodiment, the needle shaped nanocrystals extend axially from the substrate surface.

The needle shaped nanocrystals may grow from the surface of the substrate.

The needle shaped nanocrystals comprise an exemplary diameter of at least 50 nm, 55 nm, 60 nm, 65 nm, 70 nm, 75 nm, 80 nm, 85 nm, 90 nm, 95 nm, and 100 nm.

The needle shaped nanocrystals may create a mineralized layer extending from the substrate surface with a thickness of at least 1 μm, 5 μm, 10 μm, 15 μm, 20 μm, 25 μm and no more than 100 μm, 90 μm, 80 μm, 70 μm, 60 μm. The thickness may be from 1-100 μm, 10-90 μm, 20-80 μm, 30-70 μm, 40-60 μm. Preferably, the needle shaped nanocrystal layer may have a thickness of 50 μm. Thickness of the layer created by needle shaped nanocrystals is determined by the length of the nanocrystals growing axially from the substrate surface. The overall mineralized layer may be composed of multiple individual layers of needle shaped nanocrystals. As a non-limiting example, a single layer of needle shaped nanocrystals may range from about 5 μm up to about 10 μm. By growing multiple such layers on top of each other, thicknesses disclosed above are achieved.

Beneficially, these structures have been observed to have improved mechanical characteristics including stiffness. The generated layer of mineralized spiky layer is capable of being exposed without a ELP layer cover, due to the spiky layer's improved mechanical characteristics including stiffness.

### C. Flower-Like Nanocrystal Formations

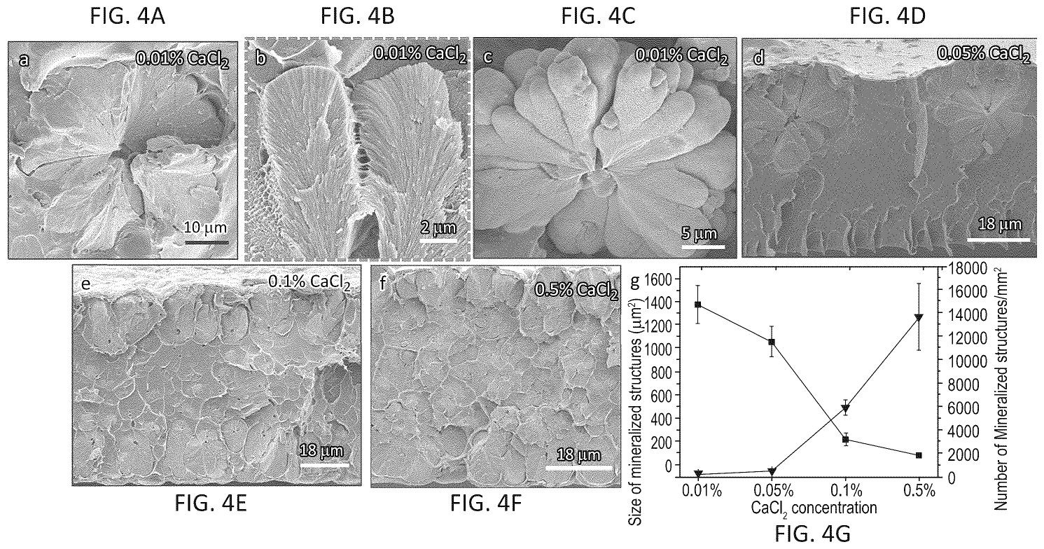

Alternatively, the crystal structure may comprise a flower-like shaped nanocrystal, as shown in FIGS. 4A-4G. Flower-like formations include bundles of hexagonal nanocrystal that grow together forming a prisms or structures similar to the “petals” of flower.

The flower-liked shaped nanocrystal may be located on the inside of the bulk of the polypeptide substrate.

Surprisingly, the inventors observed that the number of flower-liked shaped nanocrystals decreased as the percentage by weight of calcium ions incorporated into the polypeptide substrate of the present invention increased.

Surprisingly, it was observed that when flower-liked shaped nanocrystals were formed on the inside of the polypeptide substrate, needle shaped nanocrystals did not form on the exterior of the polypeptide substrate.

This demonstrates that hierarchical growth of crystals can be controlled by the use of a polypeptide substrate according to the present disclosure.

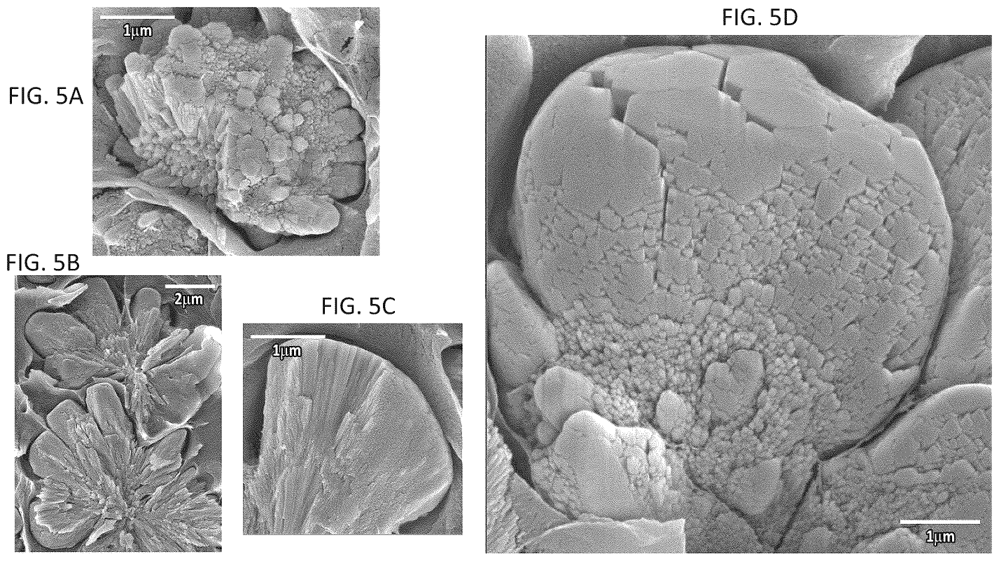

The nanocrystals within the substrate may be fused, as shown in FIGS. 5A-5D. Fusing can be achieved by mineralizing already mineralized structures (e.g., flower-like) at lower pH values (e.g., at a pH of about 4). It is additionally possible to incur fusing at pH's of about 5. At these lower pHs, mineralized structures have been shown to fuse.

Surprisingly, the inventors observed that fused crystals have improved mechanical properties and are able to fuse and better integrate with mineralized tissues.

The flower-like shaped nanocrystals comprise an exemplary diameter of at least 30 nm, 31 nm, 32 nm, 33 nm, 34 nm, and 35 nm. Diameters of flower-like nanocrystals are determined by the nanocrystals growing axially from the substrate surface in “petals.” Diameters can be increased by subjecting flower-like shaped nanocrystals to mineralization solutions for longer periods, as disclosed herein. The use of thicker substrates for growing flower-like shaped nanocrystals can also increase the diameter.

The flower-like nanocrystals may create a mineralized layer extending from the substrate surface with a thickness of at least 1 μm, 5 μm, 10 μm, 15 μm, 20 μm, 25 μm and no more than 100 μm, 90 μm, 80 μm, 70 μm, 60 μm. The thickness may be from 1-100 μm, 10-90 μm, 20-80 μm, 30-70 μm, 40-60 μm. The overall mineralized layer may be composed of multiple individual layers of flower-like nanocrystals. As a non-limiting example, a single layer of flower-like nanocrystals may range from about 2 μm up to about 20 μm. By growing multiple such layers on top of each other, thicknesses disclosed above are achieved.

The inventors observed that thicker crystals may develop inside ELP membranes having a thickness of from 0.5 mm-1.5 mm, 0.6 mm-1.4 mm, 0.7-1.3 mm, 0.8 mm-1.2 mm, or 0.9-1.1 mm.

The crystal may be antimicrobial. The term ‘antimicrobial’ generally refers to substances or components that can kill, or inhibit the growth of, microorganisms.

### V. Methods of Crystal Formation Synthesis

The present disclosure relates to methods of crystal formation synthesis. Crystals can be grown on the surface or from within the polypeptide substrates disclosed herein. As a non-limiting example, crystal formation synthesis may comprise the steps of contacting a substrate with a mineralizing solution. Crystal formation synthesis processes disclosed herein are applicable with any substrate disclosed herein. Membranes may serve as non-limiting examples in the foregoing disclosure. Additionally, elastin-like polypeptides may serve as non-limiting examples of polypeptides to be used for substrates below. It is to be understood any proteins disclosed herein can be used in the polypeptide substrates compatible for crystal growth.

The step of contacting the substrate may comprise submerging and/or incubating the substrate in the mineralizing solution. The step of incubating comprises nucleation followed by crystal growth.

The mineralizing solution may comprise PO₄³⁻ ions. As additional, non-limiting examples, mineralized structures can be grown using magnesium ions, calcium ions, phosphorus, and fluorine ions. In uses of magnesium ions, energy-dispersive x-ray spectroscopy (EDX) has shown that mineralized structures exhibited the presence of Mg indicating the incorporation of Mg ions into the crystal lattice.

The mineralizing solution may comprise at least 0.1 mM, 0.2 mM, 0.3 mM, 0.4 mM, 0.5 mM, 0.6 mM, 0.7 mM, 0.8 mM, 0.9 mM, 1 mM PO₄³⁻ ions. In some embodiments, the mineralizing solution may comprise no more than 5 mM, 4.5 mM, 4.0 mM, 3.5 mM, 3.0 mM, 2.5 mM, or 2.0 mM PO₄³⁻.

The mineralizing solution may comprise 0.1 mM-3.5 mM, 0.1 mM-3 mM, 0.1-2.5 mM, 0.2 mM-2.5 mM, 0.3-2.5 mM, 0.4-2.5 mM, 0.5-2.5 mM, 0.6-2.5 mM, 0.7-2.5 mM, 0.8-2.5 mM, 0.9-2.5 mM, 1-2.5 mM, 1.1-2.5 mM, 1.2-2.5 mM, 1.3-2.5 mM, 1.4-2.5 mM, or 1.5 mM-2.5 mM PO₄³⁻. Preferably, the mineralizing solution comprises 2 mM PO₄³⁻.

The PO₄³⁻ ions may be provided by any substance capable of donating PO₄³⁻ ions.

Preferably, the PO₄³⁻ ions are provided by calcium phosphate. Varying concentrations of calcium and phosphorus-based ions can be used in mineralizing solutions. In some instances, even saliva with very low ion concentrations can lead to mineralization. In such examples, an underlying native surface for remineralization is required. As a non-limiting example, a variety of underlying crystal structures can serve as the underlying native service. Underlying crystal structures include but are not limited to bone tissue and dental enamel. A first deposit of an ELP substrate including a coating can then be deposited and mineralized either by artificial saliva or by supersaturated mineralization solution. This will create new apatite crystals growing epitaxially from the underlying crystal structure-ELP interface outwards through the ELP matrix.

The mineralizing solution may comprise F⁻ ions.

The mineralizing solution may comprise at least 0.1 mM, 0.2 mM, 0.3 mM, 0.4 mM, 0.5 mM, 0.6 mM, 0.7 mM, 0.8 mM, 0.9 mM, 1 mM F⁻ ions. In one embodiment, the mineralizing solution may comprise no more than 5 mM, 4.5 mM, 4.0 mM, 3.5 mM, 3.0 mM, 2.5 mM, or 2.0 mM F⁻ ions.

The mineralizing solution may comprise 0.1 mM-3.5 mM, 0.1 mM-3 mM, 0.1-2.5 mM, 0.2 mM-2.5 mM, 0.3-2.5 mM, 0.4-2.5 mM, 0.5-2.5 mM, 0.6-2.5 mM, 0.7-2.5 mM, 0.8-2.5 mM, 0.9-2.5 mM, 1-2.5 mM, 1.1-2.5 mM, 1.2-2.5 mM, 1.3-2.5 mM, 1.4-2.5 mM, or 1.5 mM-2.5 mM F⁻ ions. Preferably, the mineralizing solution comprises 2 mM F⁻ ions.

The F⁻ ions may be provided by any substance capable of donating F⁻ ions.

Preferably, the F⁻ ions are provided by sodium fluoride. As additional, non-limiting examples, fluoride ions can also be provided by stannous fluoride (SnF₂) and sodium monofluorophosphate (Na₂PO₃F).

The mineralizing solution may comprise PO₄³⁻ ions and F⁻ ions.

Preferably, the mineralizing solution comprises 2 mM calcium phosphate and 2 mM sodium fluoride.

Alternatively, the mineralising solution may comprise calcium phosphate and sodium fluoride in an amount of from 0.1 mM-0.65 mM, 0.15-0.6 mM, 0.2-0.55 mM. Preferably, 0.25 mM, or 0.5 mM.

In such an embodiment, the inventors discovered that the combination of a thicker ELP membrane of around 1 mm and a concentration of calcium phosphate of around 0.1 mM-0.65 mM formed crystal structures inside the membrane having increased thickness.

The contacting step, wherein the polypeptide substrate comes into contact with a mineralizing solution, may be carried out at physiological temperature.

The contacting step may be carried out at a temperature of about 35-38° C. Most preferably, about 37° C.

The contacting step may be carried out for a period of at least 8 hours, at least 10 hours, at least 12 hours, at least 1 day, 2 days, 3 days, 4 days or 5 days. In some embodiments, the contacting step may be carried out for a period of no more than 20, 15, 10, 9 or 8 days.

The contacting step may be carried out for 1-20 days, 5-15 days, 5-10 days. The length of such step may be dependent upon the thickness of the desired substrate (e.g., membranes, particles, etc.). As a non-limiting example, a 50 μm thick membrane may require 7-10 days for an optimum mineralization period for the membranes to mineralize.

The contacting step may be carried out at a pH of at least 2, 3, 4, 5, or 6. In some embodiments, the contacting step may be carried out at a pH of no more than 11, 10, 9, or 8. The pH may be from 2-11, 3-8, 4-8, 4-7, or 5-7. Most preferably the pH is 6.

The pH may change over time. For example, the pH may decrease to around 4 after 2 days of incubation.

The present disclosure relates to methods of fusing crystal structures. Fusing typically occurs at lower pH values including but not limited to pH values of about 4 and below. At such pH values, re-mineralization of previously mineralized structures leads to fusion of adjacent crystals. Fusing can occur at elevated pH values of about 5. This produces increased thickness of individual crystals. Fusion additionally leads to improved mechanical characteristics including stiffness, cement-like characteristics, stability, acid resistance, and the like.

In such an embodiment, the process may further comprise the step of replacing the mineralizing solution. The step of replacing the mineralizing solution may be carried out after a period of 8 hours, 10 hours, 12 hours, 1 day, 2 days, 3 days, 4 days or 5 days of incubation. In doing so, the resulting crystal structure has been observed to form concentric nanocrystals inside the ELP membrane and needle shaped nanocrystals on the membrane surface as described herein.

The process may further comprise the step of maintaining the pH for the duration of the contacting step.

Preferably, the pH is maintained at pH 4-7, or 5-7, most preferably 6.

The pH may be maintained by the addition of a buffer solution.

The buffer solution may have a concentration of at least 10 mM, 15 mM, 20 mM, 30 mM and no more than 60 mM, 55 mM, 50 mM, 45 mM, 40 mM. Preferably the buffer solution has a concentration of from 25-45 mM, 30-40 mM. Most preferably, 35 mM.

Preferably the buffer solution is a BIS-TRIS buffer.

Surprisingly, the inventors discovered that maintaining the pH at 6 throughout the contact period results in the formation of flower-shaped nanocrystals on the inside of ELP membranes as described herein. More surprisingly, the inventors discovered that maintaining the pH at around 6 prevented the formation of needle shaped nanocrystals on the surface of the membrane, as described herein.

The process may further comprise the step of contacting the membrane at a first pH and then contacting the membrane at a second, lower pH. The first pH may be 5.5-6.5. The second lower pH may be from 3.0-4.0. Preferably, the first pH is around 6.0 and the second lower pH is around 4.

The membrane may be contacted at the first pH for around 7-13 days, 8-12 days, 9-11 days. Most preferably 10 days. The membrane may be contacted at the second pH for around 7-13 days, 8-12 days, 9-11 days. Most preferably 10 days.

In such an embodiment, it was observed that the crystals fused as described in.

### VI. Applications and Devices

The present disclosure relates to a variety of applications and devices of the polypeptide substrates comprising crystal formations described herein. As non-limiting examples, the present disclosure relates to applications and devices of various polypeptide substrates including crystal formations including but not limited to polypeptide particles, membranes, hydrogels, and the like including onion-like, needle-like, flower-like and other such crystal formations disclosed herein. Such combinations of polypeptide substrates and crystal formations can be used in coatings, enamels, implants, pastes, spreads, slurries, and the like.

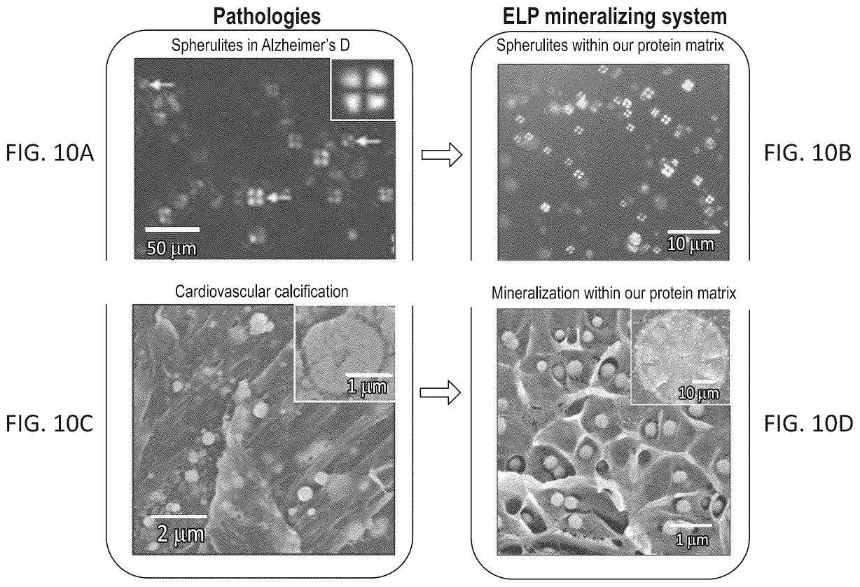

Combinations of polypeptide substrates comprising crystal formations described herein can be used in medicine, such as for use in the prevention and/or treatment of demineralisation of teeth, dental disease, dental hypersensitivity, bone demineralisation, low bone density, bone disease, bone defects, osteoporosis, or cardiovascular disease. Such combinations can additionally be used to treat diseases relating to calcification including diseases related to tissue calcification. Combinations disclosed herein are also useful for analysing and treating Alzheimer's disease. The present disclosure relates to the supramolecular organization of organic molecules that form Maltese-like cross pattern structures (organic spherulites). Such structures nucleate and grow additional mineralized structures (inorganic spherulites). Both of these organic and inorganic structures exhibit similarity to structures seen in pathologies such as cardiovascular calcification and calcification associated with Alzheimer's disease (FIGS. 10A-10D). The structures disclosed herein may present a model for exploring and understanding mechanistic insights of associated tissue calcification.

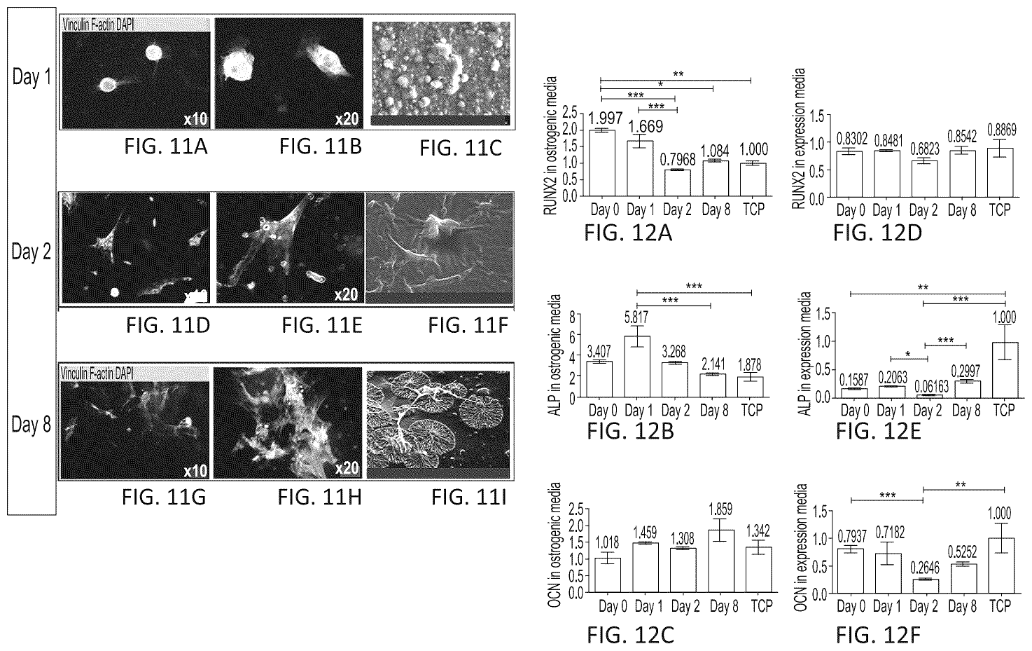

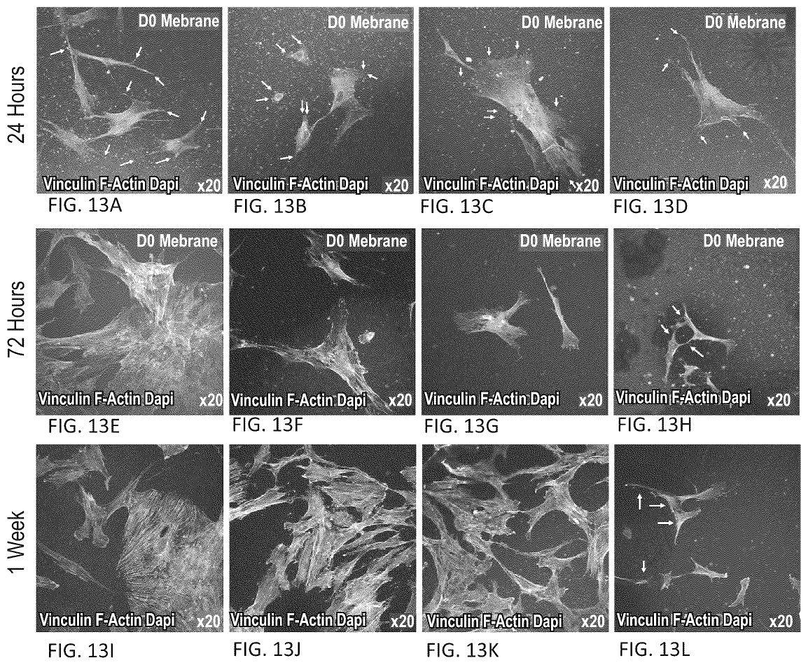

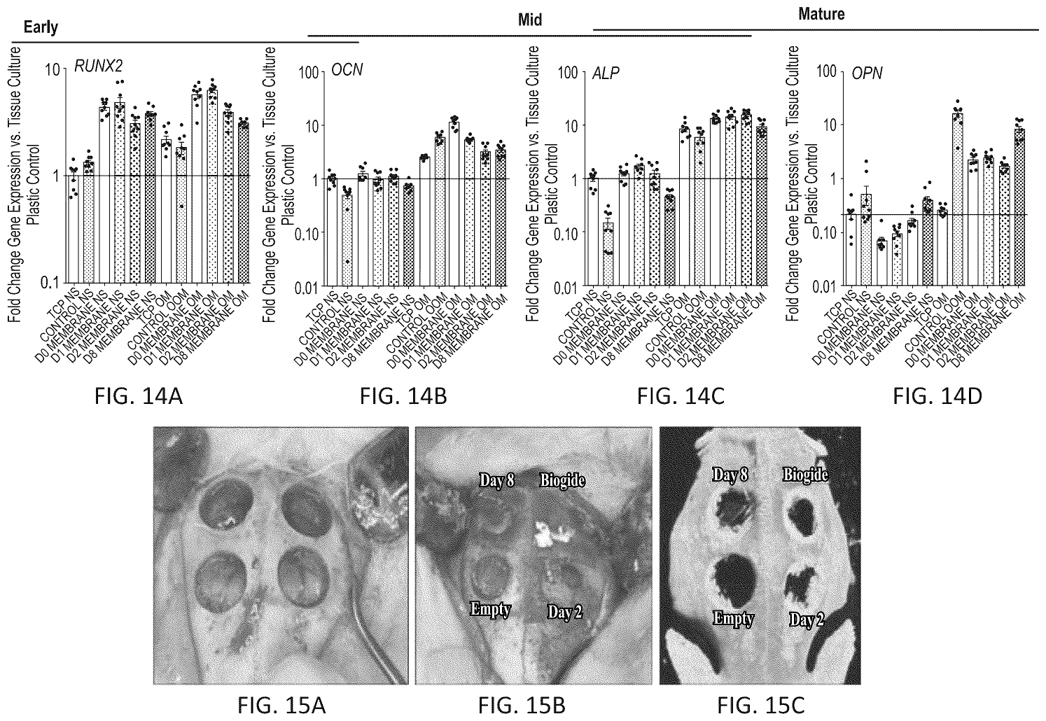

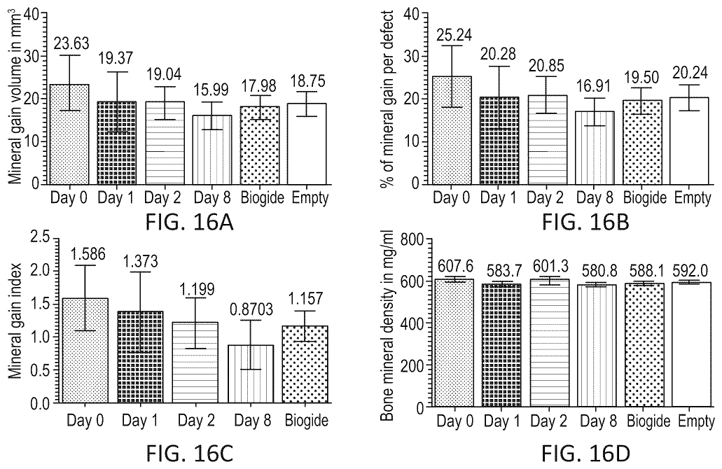

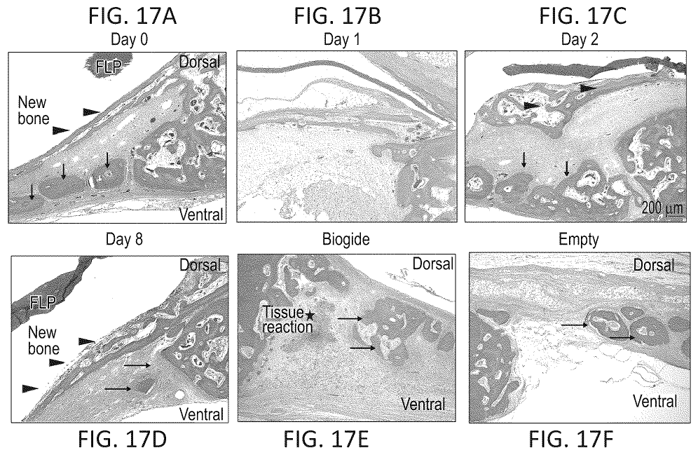

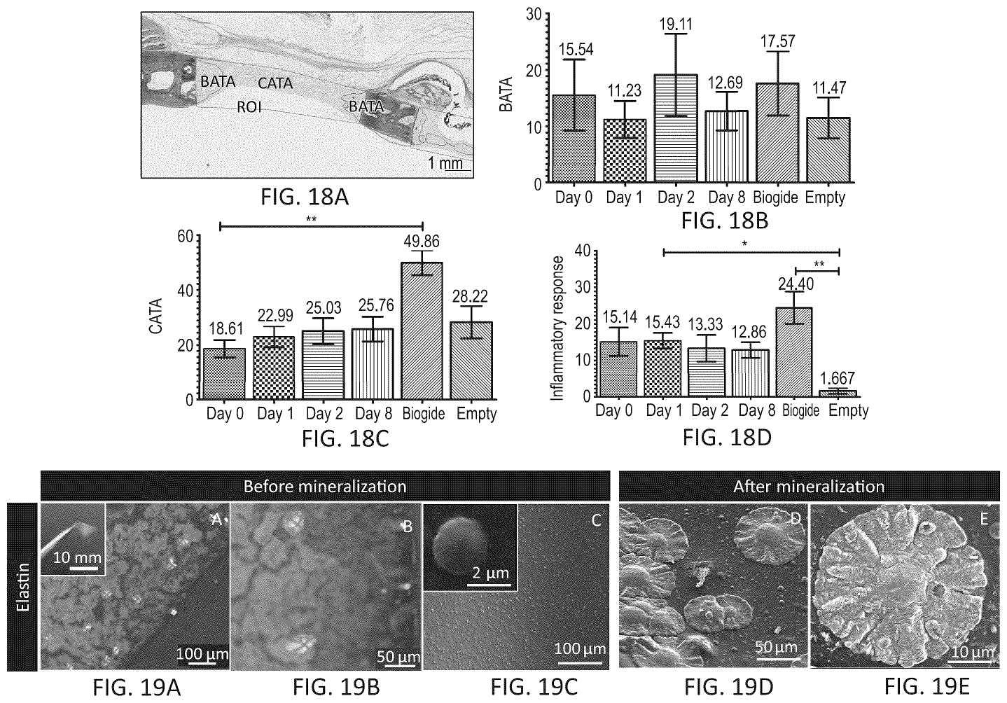

The present disclosure relates to a combination as disclosed herein for use in the prevention and/or treatment of bone demineralisation, low bone density, bone disease, bone defects and/or osteoporosis. FIGS. 11A-18D display both in vitro results (FIGS. 11A-14D) and in vivo results using rabbits (FIGS. 15A-18D) of using combinations disclosed herein to treat bone defects. Combinations disclosed herein can additionally be used in treating or preventing cardiovascular disease.

The present disclosure relates to a medical device or pharmaceutical composition comprising a combination, as disclosed herein. The medical device may be a medical implant, synthetic graft, prosthesis, orthosis, paste, malleable putty, film, or bone implant.

The present disclosure relates to a medical device or pharmaceutical composition comprising a substrate, as disclosed herein. The medical device may be a medical implant, synthetic graft, prosthesis, orthosis, paste, malleable putty, film, or bone implant.

The present disclosure relates to a crystal for use in the prevention and/or treatment of demineralisation of teeth, dental disease, dental hypersensitivity, bone demineralisation, low bone density, bone disease, bone defects, osteoporosis, or cardiovascular disease.

The present disclosure relates to a substrate for use in the prevention and/or treatment of demineralisation of teeth, dental disease, dental hypersensitivity, bone demineralisation, low bone density, bone disease, bone defects, osteoporosis, or cardiovascular disease.

The present disclosure relates to a substrate as described herein for use in a method of tissue regeneration, the method comprising the steps of depositing the substrate on the tissue and contacting the substrate with a mineralization solution.

The tissue may be any tissue with an underlying crystal structures, such as bone tissue.

Substrates including various crystal growths, as disclosed herein, may be coated or partially coated on a medical implant, synthetic graft, prosthesis, orthosis, paste, hydrogel, malleable putty, film, three-dimensional printed implants, or bone implant. The surface of the medical implant, synthetic graft, prosthesis, orthosis, paste, malleable putty, film, dental implant or bone implant may be partially or fully covered with the crystal and/or substrate. The coating can be chemically bonded to the surface through a variety of mechanisms including but not limited to covalent bonding, physisorption, and the like.

In a further aspect of the present invention there is provided a method of growing a crystal according to the present disclosure on a medical implant, synthetic graft, prosthesis, orthosis, paste, malleable putty or film, the method comprising the steps of contacting a medical implant, synthetic graft, prosthesis, orthosis, paste, malleable putty or film comprising a substrate with a mineralizing solution as described herein. As such, the medical device or material can grow various crystal structures from the substrate contained therein.

### VII. Methods of Use in Cardiovascular Calcification

The present disclosure relates to uses of disclosed compositions of polypeptide substrates and crystal formations to assess and treat tissue calcification. In particular, the present disclosure relates to cardiovascular calcification. Compositions disclosed herein help to illuminate the mechanisms behind cardiovascular calcification. Though traditional mechanisms focus on cellular processes leading to or controlling the unwanted mineralization on soft tissues, extracellular components including elastin are fundamental in regulating the mechanical properties of heart tissues.

The present disclosure relates to a toolkit to control compositions of tissues by selective digestion of ECM components. This can be used in designing disease-specific in-vitro models, as disclosed herein. Systematic enzymatic digestion of cardiovascular tissues illuminates elastin's role in cardiovascular calcification. Elastin's degeneration displays evident changes in the structure and composition of extracellular matrix (ECM) of heart tissue. As such, the present disclosure relates to methods of treatment and prevention of unwanted pathological disorders.

Human aorta is made from three layers known as tunica adventitia, tunica media, and tunica intima, which are all responsible for circulating oxygenated blood from the heart throughout the whole body. Tsamis, A., J. R. Soc. Interface 2013, 10 (83), 20121004; Komutrattananont, P., et al., Anat. Cell Biol. 2019, 52 (2), 109-114. Within this microstructure of the aortic wall, elastin and collagen are the main contributors to its elasticity and mechanical strength, respectively. Berillis, P., Open Circ. Vasc. J. 2013, 6 (1). Collagen is mainly located in the tunica adventitia (outer layer) and tunica media (middle layer), while elastin is mainly located in tunica media (middle layer). In cardiovascular diseases, inflammatory conditions can affect the compliance of the aortic wall, which can be observed by changes in the aorta's diameter, length, and thickness, whereas age-related changes cause enlargement and structural changes in tunica media. Komutrattananont, P., et al., Anat. Cell Biol. 2019, 52 (2), 109-114; Berillis, P., Open Circ. Vasc. J. 2013, 6 (1).

Collagen a component of the extracellular matrix (ECM) of heart valves, providing stiffness, strength, and stability of the valve's cusps. Kodigepalli, K. M., et al., J. Cardiovasc. Dev. Dis. 2020, 7 (4), 57. Elastic fibers, which are mainly composed of elastin, are predominantly arranged in the form of continuous sheets along the radial and circumferential axes, which facilitate valve motion and bear a substantial amount of load without deformation. Hinton, R. B., et al., Annu. Rev. Physiol. 2011, 73, 29-46. In a diseased environment, the cusp and leaflet of the valves thickens as a result of changes in the organization of collagen fibers and the emergence of calcification.

While molecular mechanism of pathological calcification remains unclear, several studies have highlighted events resembling those in bone formation. Kempf, H., et al., Front. Cell Dev. Biol. 2021, 9; Tintut, Y., et al., Biomolecules 2021, 11 (10), 1482. For instance, in vessel wall calcification, some cellular processes resemble those of developmental osteogenesis including competition between mineralization inhibitors and promoters, osteoblastic differentiation, expression of bone matrix proteins, and formation of hydroxyapatite. Kapustin, A., Curr. Opin. Pharmacol. 2009, 9 (2), 84-89; Persy, V. P., et al., Kidney Int. 2011, 79 (5), 490-493; Herrmann, M., et al., PLOS One 2020, 15 (2), e0228503; Kaartinen, M. T., et al., J. Histochem. Cytochem. 2007, 55 (4), 375-386; Rajamannan, N. M., et al., Circulation 2011, 124 (16), 1783. Moreover, studies have reported the presence of calcifying osteoblast-like cells within human aortic valve cell cultures in vitro. Yu, B., et al., Basic to Transl. Sci. 2017, 2 (4), 358-371. Furthermore, calcified particles have been linked to triggering osteoblastic differentiation of mesenchymal stem cells linked to vascular tissue, but the source of these calcified particles has not been identified. Several studies have taken a materials science approach to shine light on this mysterious process. Bertazzo, S., et al., Nat. Mater. 2013, 12 (6), 576-583; Hutcheson, J. D., et al., Nat. Mater. 2016, 15 (3), 335-343. In a pioneering study, Stevens and colleagues used advanced nano-analytical microscopy techniques and found that the unset of cardiovascular calcification is not associated to surface precipitation of calcium phosphate, but rather a more complex biomineralization process that occurs within the bulk of the tissue. Radvar, E., et al., Adv. NanoBiomed Res. 2021, 1 (8), 2100042.

The ECM is of paramount importance in the formation of calcified structures. Even in the absence of cells, tissues can become calcified. Watson, K. E., et al., Arterioscler. Thromb. Vasc. Biol. 1998, 18 (12), 1964-1971. Collagen is believed to be one of the main sources of extracellular calcification in cardiovascular tissues in a process that progresses slowly over years or decades, resulting in accumulation of collagen, calcification, and disruption of the tissue microarchitecture. Ruiz, J. L., et al., Cardiovasc. Pathol. 2015, 24 (4), 207-212. However, these studies have focused on analyzing tissues at later stages of calcification and have not addressed the initial events triggering this process. In addition, mineral-associated vesicles (MVs) from cells undergoing osteoblastic differentiation can nucleate and grow hydroxyapatite crystals when bound to not only collagen, but also other ECM components such glycosaminoglycans (GAGs). Kapustin, A. N., et al., Circ. Res. 2011, 109 (1), e1-e12. Similarly, the source of these MVs has not been identified. Furthermore, the apparent participation of other ECM components in calcification confirms a complex multifactorial calcification scenario, which underlines the importance of identifying the origins of the calcification process.

Elastin is the predominant ECM component of elastic fibers in cardiovascular connective tissues. Elastic fibers possess a very low turnover rate and thus insult to elastic tissue can result in either degradation due to chronic loss or excess (detrimental) accumulation. Humphrey, J. D., et al., Nat. Rev. Mol. cell Biol. 2014, 15 (12), 802-812; Bailey, E. L., et al., Atherosclerosis 2014, 237 (2), e4. During the initial stages of cardiovascular calcification, macrophage derived elastolytic enzymes and matrix metalloproteinases degrade elastin, resulting in the release of soluble elastin-derived peptides that can promote osteogenic differentiation and subsequent calcification. Bailey, M., Cardiovasc. Pathol. 2004, 13 (3), 146-155; Green, E. M., et al., Interface Focus 2014, 4 (2), 20130058. Sakata et al. reported that modification in the elastin content of aorta led to calcification in the aortic media. Sakata, N., et al., Nephrol. Dial. Transplant. 2003, 18 (8), 1601-1609. Also, in atherosclerotic aorta, both apatite and whitlockite-type minerals are shown by Raman spectroscopy to localize in the tunica media (the elastic layer) as reported by You et al. in 201727. Moreover, it has been shown that elastin can be associated to matrix vesicles and has a greater propensity for calcification as a result of ageing and specific pathologies (REF). The role of elastin in inducing mineralization is reported in a few studies in vitro. Kapustin, A. N., et al., Circ. Res. 2011, 109 (1), e1-e12; Parashar, A., et al., J. Struct. Biol. 2021, 213 (1), 107637. For example, Gourgas et al. reported the deposition of globular calcium phosphate minerals on fibers and filaments on elastin-like polypeptide (ELP) membranes. Gourgas, O., et al., Biomacromolecules 2019, 20 (7), 2625-2636. Furthermore, the present disclosure relates to methodologies to engineer ELP-based membranes with tunable ELP conformation and geometrical confinement to investigate organic-inorganic interactions within bulk environments. Tejeda-Montes, E., et al., Acta Biomater. 2012, 8 (3), 998-1009; Tejeda-Montes, E., Biomaterials 2014, 35 (29), 8339-8347; Elsharkawy, S., et al., Nat. Commun. 2018, 9 (1), 2145; Deng, X., et al., Mater. Today Bio 2021, 11, 100119.

There is currently no definitive therapy to prevent or treat cardiovascular calcification and the underlying mechanisms triggering this condition are not fully understood. The risk factors rare not consistently correlated, leaving clinicians uncertain about the optimum management for these patients34. Surgery is the only effective treatment but could lead to damage of the aortic or mitral valves, which would require surgical replacement of the valve. In addition, bio-prosthetic valves still carry a high risk of calcification and operative mortality. As such, the present disclosure relates to uses of compositions disclosed herein in order to illuminate the role of elastin in cardiovascular calcification, as well as treating and preventing such calcification.

The present disclosure relates to a major role played by elastin in the onset of calcification of cardiovascular tissues. Furthermore, materials science techniques known in the art are used to characterize aorta and mitral valve tissues at multiple size-scales from the molecular scale and found that elastin plays a bigger role sequestering Ca²⁺ ions and generating Ca²⁺-based mineralized structures compared to collagen. This demonstrates an enhanced propensity to calcify.

The present disclosure relates to elastin degradation and accumulation leading to changes in the structure and composition of the ECM and generating a supramolecular framework that can promote mineralization. Despite mounting evidence that elastin may be central to the onset and progression of cardiovascular calcification, limited attention has been given at understanding its contribution from a structural standpoint and from the molecular scale. The present disclosure relates to this critical role of elastin.

### ASPECTS

The present disclosure is related to the following aspects.
1. A polypeptide substrate comprising calcium ions, wherein the calcium ions are embedded within the polypeptide substrate.
2. A substrate according to aspect 1 wherein the calcium ions are Ca²⁺.
3. A substrate according to any preceding aspect, wherein the calcium ions are provided by CaCl₂).
4. A substrate according to any preceding aspect, wherein the calcium ions are present in an amount of at least 0.001% by weight of the substrate.
5. A substrate according to aspect 4, wherein the calcium ions are present in an amount of at least 0.005-1.5% by weight of the substrate.
6. A substrate according to any preceding aspect, wherein the polypeptide is a pentapeptide Elastin-like-polypeptide selected from the group consisting of Gly-X-X-X-X, X-Gly-X-X-X, X-X-Gly-X-X, X-X-X-Gly-X and X-X-X-X-Gly, (GXXXX, XGXXX, XXGXX, XXXGX, XXXXG), wherein X is any amino acid from apart proline or

MGSSHHHHHHSSGLVPRGSHMESLLP-
[((VPGIG)2VPGKG(VPGIG)2)2-
DDDEEKFLRRIGRFG-((VPGIG)2VPGKG(VPGIG)2)2]3-V.

7. A substrate according to any preceding aspect, wherein the polypeptide substrate has a thickness of from 0.5 mm-1.5 mm.
8. A process for forming an elastin-like polypeptide membrane according to any one of aspects 1 to 7, the process comprising the steps of:
a) dissolving elastin-like polypeptides with a source of calcium ions and a solvent to form an ELP solution; and
b) applying the solution onto a surface to form a membrane.
9. A process according to aspect 8, wherein the ELP is present in an amount of from 1-20% by weight of the solution.
10. A process according to aspect 8 or 9, wherein the source of calcium ions may be present in an amount of 0.005-1.5% by volume of the solution.
11. A process according to any one of aspects 8 to 10, wherein the step a) further comprises the step of mixing the ELP solution with a cross-linker.
12. A process according to aspect 11, wherein the cross-linker is hexamethyl diisocyanate.
13. A crystal formed from and at least partly embedded in a substrate according to any one of aspects 1 to 7.
14. A crystal according to aspect 13, wherein the crystal is located at least partly inside the bulk of the polypeptide substrate.
15. A crystal according to aspect 13, wherein the crystal is located partly inside the bulk of the polypeptide substrate and partly on the surface of the polypeptide substrate.
16. A crystal according to any one of aspects 13 to 15, wherein the crystal has a hierarchical structure.
17. A crystal according to any one of aspects 13 to 16, wherein the crystal comprises nanocrystals.
18. A crystal according to aspect 17, wherein the nanocrystals are arranged in concentric layers.
19. A crystal according to aspect 17, wherein the nanocrystals have a needle shape.
20. A crystal according to aspect 19, wherein the needle shaped nanocrystals are located on the polypeptide substrate surface and orientated perpendicular to the polypeptide substrate surface.
21. A crystal according to aspect 17, wherein the nanocrystal have a flower-liked shaped.
22. A crystal according to any one of aspects 17 to 21, wherein the nanocrystals within the substrate are fused.
23. A process for producing a crystal according to any one of aspects 13 to 22 comprising the steps of contacting a substrate according to any one of aspects 1 to 8 with a mineralizing solution.
24. A process according to aspect 23, wherein the mineralizing solution comprises PO₄³⁻ ions and F⁻ ions.
25. A process according to aspect 24, wherein the PO₄³⁻ ions and F⁻ ions may be present in a concentration of 1 mM-3 mM.
26. A process according to aspect 24, wherein the PO₄³⁻ ions and F⁻ ions may be present in a concentration of 0.1 mM-0.65 mM.
27. A process according to any one of aspects 23 to 26, wherein the contacting step is carried out for a period of 5-10 days.
28. A process according to any one of aspects 23 to 27, wherein the pH of the contacting step is from 4-7.
29. A process according to any one of aspects 23 to 28, further comprising the step of replacing the mineralizing solution after 2 days of incubation.
30. A process according to any one of aspects 23 to 29, further comprising the step of maintaining the pH for the duration of the contacting step, wherein preferably the pH is maintained at pH 4-7.
31. A process according to any one of aspects 23-27, further comprising the steps of contacting the substrate at a first pH and then contacting the substrate at a second, lower pH, wherein preferably the first pH is from 5.5-6.5 and the second lower pH is from 3.0-4.0.
32. A crystal according to any one of aspects 13-22 for use in medicine, such as for use in the prevention and/or treatment of demineralisation of teeth, dental disease, dental hypersensitivity, bone demineralisation, low bone density, bone disease, bone defects, osteoporosis, or cardiovascular disease.
33. A medical device, such as a medical implant, synthetic graft, coating, prosthesis, orthosis, paste, malleable putty, film, bone implant, or pharmaceutical composition, comprising a crystal according to any one of aspects 13 to 22.
34. A membrane according to any one of aspects 1 to 7 for use in medicine, such as for use in the prevention and/or treatment of demineralisation of teeth, dental disease, dental hypersensitivity, bone demineralisation, low bone density, bone disease, bone defects, osteoporosis, or cardiovascular disease.
35. A medical device, such as a medical implant, synthetic graft, prosthesis, orthosis, paste, malleable putty, film, bone implant, or pharmaceutical composition comprising a substrate according to any one of aspects 1 to 7.
36. A method of growing a crystal according to any one of aspects 13 to 22 on a medical implant, synthetic graft, prosthesis, orthosis, paste, malleable putty or film, the method comprising contacting a medical implant, synthetic graft, prosthesis, orthosis, paste, malleable putty or film comprising a substrate according to any one of aspects 1 to 7 with a mineralizing solution.
37. A hybrid organic-inorganic system comprising:
ordered apatite crystalline structures,
wherein the ordered apatite crystalline structures comprise at least one of onion-like, needle-like, flower-like, and prism-like microstructures; and
a protein substrate capable of growing the ordered apatite crystalline structures thereon or therein,
wherein the protein substrate comprises a polypeptide,
wherein the polypeptide comprises a pentapeptide elastin-like-polyptide (ELP) selected from the group consisting of Gly-X-X-X-X, X-Gly-X-X-X, X-X-Gly-X-X, X-X-X-Gly-X and X-X-X-X-Gly, (GXXXX, XGXXX, XXGXX, XXXGX, XXXXG), wherein X is any amino acid apart from proline,
wherein the protein substrate comprises an amount of ions embedded in the protein substrate, and
wherein the ions embedded in the protein substrate provide nucleation points to allow for selectively tailoring the ordered apatite crystalline structures.
38. A hybrid organic-inorganic system according to aspect 37, wherein the ions are selected from a group consisting of calcium ions, fluoride ions, and zinc oxide ions.
39. A hybrid organic-inorganic system according to aspect 37 wherein the ions comprise calcium ions, and wherein the calcium ions comprise Ca²⁺.
40. A hybrid organic-inorganic system according to aspect 37, wherein the ions are provided by CaCl₂).
41. A hybrid organic-inorganic system according to aspect 37, wherein the ions are present in an amount of at least 0.001% by weight of the substrate.
42. A hybrid organic-inorganic system according to aspect 41, wherein the ions are present in an amount of at least 0.005-1.5% by weight of the substrate.
43. A hybrid organic-inorganic system according to aspect 37, wherein the protein substrate has a thickness of from 0.5 mm-1.5 mm.
44. A hybrid organic-inorganic system comprising:
ordered apatite crystalline structures,
wherein the ordered apatite crystalline structures comprise at least one of onion-like, needle-like, flower-like, and prism-like microstructures; and a protein substrate capable of growing the ordered apatite crystalline structures thereon or therein,
wherein the protein substrate comprises a polypeptide,
wherein the polypeptide comprises a pentapeptide elastin-like-polyptide (ELP) selected from the group consisting of MGSSHHHHHHSSGLVPRGSHMESLLP-[((VPGIG)₂VPGKG(VPGIG)₂)₂-DDDEEKFLRRIGRFG-((VPGIG)₂VPGKG(VPGIG)₂)_{2]3}-V,
wherein the protein substrate comprises an amount of ions embedded in the protein substrate, and
wherein the ions embedded in the protein substrate provide nucleation points to allow for selectively tailoring the ordered apatite crystalline structures.
45. A hybrid organic-inorganic system according to aspect 44, wherein the ions are selected from a group consisting of calcium ions, fluoride ions, and zinc oxide ions.
46. A hybrid organic-inorganic system according to aspect 44 wherein the ions comprise calcium ions, and wherein the calcium ions comprise Ca²⁺.
47. A hybrid organic-inorganic system according to aspect 44, wherein the ions are provided by CaCl₂).
48. A hybrid organic-inorganic system according to aspect 44, wherein the ions are present in an amount of at least 0.001% by weight of the substrate.
49. A hybrid organic-inorganic system according to aspect 48, wherein the ions are present in an amount of at least 0.005-1.5% by weight of the substrate.
50. A hybrid organic-inorganic system according to aspect 44, wherein the protein substrate has a thickness of from 0.5 mm-1.5 mm.

### EXAMPLES

### Membrane Formation:

### Example 1

An ELP and calcium chloride were dissolved in anhydrous dimethylformamide (DMF) and dimethyl sulfoxide (DMSO) in order to form a ELP solution. The ELP was present in an amount of 5% by weight of the solution and the calcium chloride was present in an amount of 0.01% by weight of the solution. The ratio of DMF and DMSO was 9:1. By way of non-limiting example, the ELP may be or comprise a statherin-ELP. As another example, the ELP may be or comprise i) a pentapeptide sequence selected from the group consisting of Gly-X-X-X-X, X-Gly-X-X-X, X-X-Gly-X-X, X-X-X-Gly-X and X-X-X-X-Gly, (GXXXX, XGXXX, XXGXX, XXXGX, XXXXG), wherein X is any amino acid apart from proline, (ii) RGDS-ELP, and/or (iii) Statherin-ELP. As a further example, the ELP may consist of or comprise the sequence

MGSSHHHHHHSSGLVPRGSHMESLLP-
[((VPGIG)2VPGKG(VPGIG)2)2-
DDDEEKFLRRIGRFG-((VPGIG)2VPGKG(VPGIG)2)2]3-V.

The ELP solution was then mixed with a cross-linking solution comprising hexamethyl diisocyanate (HDI) and the solutions were drop casted on a PDMS surface. The solutions were then left to dry overnight at room temperature in a low-humidity conditions (less than 20%) inside a polymer glove box.

### Example 2

An ELP and calcium chloride were dissolved in DMF and DMSO to form an ELP solution. The ELP was present in an amount of 5% by weight of the solution and the calcium chloride was present in an amount of 0.05% by weight of the solution. The ratio of DMF and DMSO was 9:1.

The ELP solution was then mixed with a cross-linker solution comprising HDI and the solutions were drop casted on a PDMS surface. The solution was then left to dry overnight at room temperature in a low-humidity conditions (less than 20%) inside a polymer glove box.

### Example 3

An ELP and calcium chloride was dissolved in anhydrous DMF and DMSO to form an ELP solution. The ELP was present in an amount of 5% by weight of the solution and the calcium chloride was present in an amount of 0.1% by weight of the solution. The ratio of DMF and DMSO was 9:1.

The ELP solution was then mixed with a cross-linker solution comprising HDI and the solutions were drop casted on a PDMS surface. The solution was then left to dry overnight at room temperature in a low-humidity conditions (less than 20%) inside a polymer glove box.

### Example 4

An ELP and calcium chloride were dissolved in anhydrous DMF and DMSO to form an ELP solution. The ELP was present in an amount of 5% by weight of the solution and the calcium chloride was present in an amount of 0.5% by weight of the solution. The ratio of DMF and DMSO was 9:1.

The ELP solution was then mixed with a cross-linker solution comprising HDI and the solutions were drop casted on a PDMS surface. The solution was then left to dry overnight at room temperature in a low-humidity conditions (less than 20%) inside a polymer glove box.

### Example 5

An ELP and calcium chloride were dissolved in anhydrous DMF and DMSO to form an ELP solution. The ELP was present in an amount of 5% by weight of the solution and the calcium chloride was present in an amount of 0.5% by weight of the solution. The ratio of DMF and DMSO was 9:1.

The ELP solution was then mixed with a cross-linker solution comprising HDI and the solutions were drop casted on a PDMS surface. The solution was then left to dry overnight at room temperature in a low-humidity conditions (less than 20%) inside a polymer glove box. The resulting ELP membrane had a thickness of 1 mm.

### Crystal Formation:

### Example 6: Onion-Like Shaped Nanocrystals

The membranes of examples 1-4 were incubated in a mineralizing solution of 2 mM of hydroxyapatite powder and 2 mM of sodium fluoride at 37° C. The incubation was carried out at a pH of 6.0 for a period of 30 days. After 2 days, the pH decreased to around 4 and so the mineralizing solution was replaced every 2 days in order to restore the pH to 6.0.

By replenishing mineralizing solution every 2 days, additional layers of minerals grew on top of the previous mineralized layer. This grew multiple layers of crystals on top of each other to create an onion-like structure. The membranes developed a number of onion-like shaped nanocrystals in the inside of the membrane as shown in FIGS. 1A-1E.

As seen in FIG. 2H, the number of onion-like nanocrystals decreased as the percentage by weight of calcium chloride in the membrane increased. For example, the crystal formed from the membrane described in Example 4 showed the lowest number of onion-like crystals. In contrast, Example 1 showed the highest number of onion-like crystals.

In addition, as shown in FIGS. 3A-3B, the membranes also formed needle shape nanocrystals extending perpendicularly from the membrane surface. After 30 days of incubation the needle shaped nanocrystals exhibited a thickness of around 50 μm. The structures also had improved mechanical characteristics including stiffness compared to existing nanocrystals formed from ELP membranes and a stiffness more similar to natural dental enamel.

### Example 7: Flower-Liked Shaped Nanocrystals

The membranes of examples 1-4 were incubated in a mineralizing solution of 2 mM of hydroxyapatite powder and 2 mM of sodium fluoride at 37° C. The incubation was carried out at a pH of 6.0 for a period of 30 days. The pH was controlled throughout the incubation period by the addition of 35 mM Bis-Tri buffer. The mineralizing solution was replenished after every 6 days.

The membranes developed a number of flower-liked shaped nanocrystals in the inside of the membrane as shown in FIGS. 4A-4F.

As seen in FIG. 4G, the number of flower-liked shaped nanocrystals increased, and their size decreased as the percentage by weight of calcium chloride in the membrane increased. For example, the crystal formed from the membrane described in Example 4 showed the highest number of flower-liked shaped crystal and the smallest flower-liked shaped nanocrystals. In contrast, Example 1 showed the lowest number of flower-liked shaped crystals and the largest flower-liked shaped nanocrystals.

Surprisingly, as shown in FIGS. 4A-4F, the surface of the membranes did not show any needle shaped nanocrystals on the surface of the membrane as seen in Example 5.

### Example 8: Crystal Fusion Inside the Membrane

The membranes of examples 1-4 were incubated in a mineralizing solution of 2 mM of hydroxyapatite powder and 2 mM of sodium fluoride at 37° C. The incubation was carried out at a pH of 4 for a period of 20 days.

As shown in FIGS. 5A-5D, a number of crystals fused inside the membrane.

### Example 9: Formation of Crystals Inside Thicker Membranes

The membrane of example 5 was incubated in a mineralizing solution of 0.25 mM of hydroxyapatite powder and 0.25 mM of sodium fluoride at 37° C. The incubation was carried out at a pH of 6.0 for a period of 30 days.

The membrane developed crystal structures inside the membrane. These crystal structures were observed to have an increased thickness, as shown in FIGS. 6A-6D.

### Example 10: Formation of Crystals Inside Thicker Membranes

The membrane of example 5 (comprising 0.5% CaCl₂) and 5% ELP having a thickness of 1 mm) was incubated in a mineralizing solution of 0.5 mM of hydroxyapatite powder and 0.5 mM of sodium fluoride at 37° C. The incubation was carried out at a pH of 6.0 for a period of 30 days.

The membrane developed crystal structures inside the membrane. These crystal structures were observed to have an increased thickness.

Hydroxyapatite crystals were separately grown by omitting fluoride ions in the mineralization solution.

### Example 11: Coating of 3D Printed Nylon and Titanium Substrates

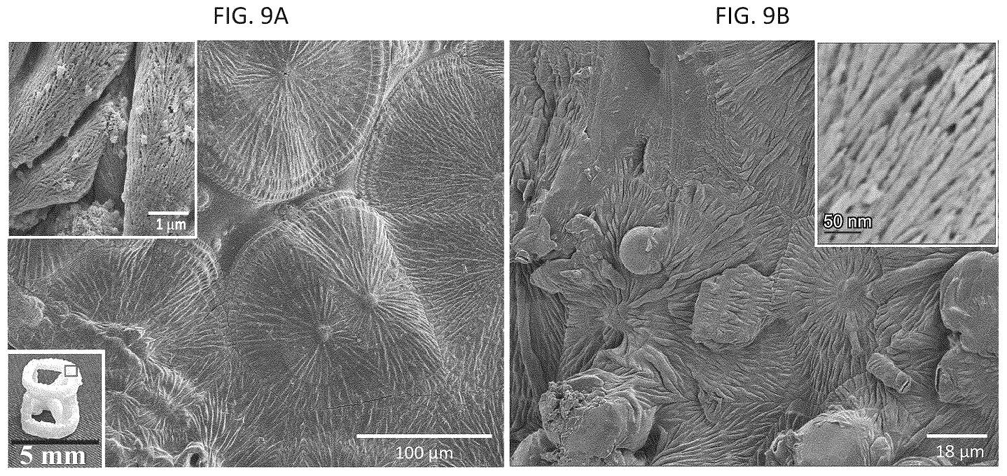

Substrates of titanium and nylon were dipped in a membrane solution of example 1 and placed on PDMS surface for drying overnight in a glove box. SEM analysis of the coated substrates revealed formation of 10 μm thick coatings formed uniformly over a large surface area, as shown in FIGS. 9A-9B. The coated substrates were then placed in a beaker containing mineralizing solution of 2 mM hydroxyapatite and 2 mM sodium fluoride 2 mM at pH 6 and 37° C. for 5, 10, and 15 days. The resulting substrates were analysed for the formation of crystal structures using SEM, as shown in FIGS. 9A-9B.

### Example 12: Cardiovascular Calcification

### Methods and Materials

### Elastin Membrane Preparation

Membranes were fabricated by dissolving commercially available natural elastin (bovine neck ligament, Elastin Products Company—EPC, USA) in 90% anhydrous dimethylformamide (DMF, Sigma Aldrich, Merck, Germany) and 10% Dimethyl sulfoxide (DMSO, Sigma Aldrich, Merck, Germany) at room temperature in a low-humidity conditions (less than 20%) inside a glove box. In order to crosslink the proteins, hexamethyl diisocyanate (HDI, Sigma Aldrich, Merck, Germany) was added to protein solutions for crosslinking at a ratio of 1:3 (lysine to HDI) and drop-casted on top of Polydimethylsiloxane (PDMS, Sylgard™ 184 Silicone Elastomer Kit, Dow, USA) substrate, left to dry overnight. Formed membranes were washed with deionized water, then they were observed under polarized light microscope (VHX-S750E, Keyence) with a cross-polarizer to check the formation of elastin spherulites.

### Animal Tissues

Aorta and mitral valve tissue was harvested from a bovine heart supplied by a local slaughterhouse. The aorta samples had a mass of between 45.98-73.42 mg and the mitral valves a mass of 0.41-9.24 mg. The harvested aorta and mitral valve samples were stored at −20° C. until use.

### Enzymatic Degradation

Aorta and mitral valve were separated from bovine heart tissue and were digested by elastase (19.4 U/mg, Elastin Products Company—EPC, USA) at a concentration of 1.37 U/ml in 1× phosphate buffered saline (PBS, Sigma Aldrich, Merck, Germany). Tissues were also digested in collagenase (125 U/mg, Elastin Products Company—EPC, USA) at a concentration of 400 U/ml in 1×PBS. The samples were incubated in enzyme solution for 1 and 3 hours at 37° C. Later, they were washed in 1×PBS followed by ultra-pure water for 20 seconds.

### Mineralization

The mineralization solution containing hydroxyapatite (2 mM) and sodium fluoride (2 mM) were prepared in deionized water under continuous stirring. Subsequently, 69% (v/v) nitric acid was added dropwise until the powder was completely dissolved. Later, the pH of the solution was adjusted to 6.0 by adding 30% (v/v) ammonium hydroxide (Sigma Aldrich, Merck, Germany) solution. Elastin and collagen membranes were placed in mineralization solution (50 ml) and incubated for eight days at 37° C. using a temperature-controlled incubator (LTE Scientific, Oldham, UK).

### Scanning Electron Microscopy (SEM)

Samples were mounted on aluminium stubs after being dried via self-adhesive tape and were coated using an auto sputter coating machine with a conductive material. Samples were analyzed using an FEI Inspect F (Hillsboro, USA). Their surface topography was observed using a secondary electron detector. A BSE detector was used to assess the variation in density within each sample. In other instances, samples were investigated using SEM (Gemini 1525 FEGSEM), operated at 10 kV. The instrument was equipped with both an inlens detector that recorded secondary electrons, and a backscatter electron detector. The DDC-SEM images were obtained by imaging the same region with both inlens mode and backscatter mode. Using ImageJ software, both images were stacked and the inlens image was assigned to the green channel whereas the backscatter image was assigned to the red channel following the technique reported in Bertazzo et al. 18. Collagen membranes were mounted on carbon tape and coated with gold layer using sputter coating (Leica EM ACE600) and were imaged by JEOL NeoScope JCM 6000Plus (JEOL Ltd., Tokyo, Japan).

### Transmission Electron Microscopy (TEM)

Aorta tissue was imaged by TEM to analyze its collagen and elastin content. Tissue sample was embedded in Araldite resin at room temperature then was cut using a Ultramicrotome Reichert-Jung E (Leica). After mounting on a grid, sections were post stained with 2% Uranyl Acetate and Reynold's Lead Citrate according to standard method. Bright-field TEM imaging was performed on a JEM-1230 TEM (JEOL Ltd., Tokyo, Japan) operated at an acceleration voltage of 80 kV, and the images were recorded by Morada camera with iTEM software (Olympus-EMSIS).

### Histological Analysis

Digested aorta and mitral valve samples were embedded in paraffin wax blocks, cut into sections of about 3 μm, and stained with Elastin Von Gieson (EVG), Von Kossa and Masson Trichrome (MT) to visualize under optical light microscope its elastin content, calcium deposits and collagen content, respectively.

### Results

### In-Vitro Models of Elastin

In order to investigate the capacity of elastin in directing mineralization, an in-vitro model approach was used, which has been reported in previous studies to compare the mineralization pattern in elastin. Gourgas, O., et al., Biomacromolecules 2019, 20 (7), 2625-2636; Elsharkawy, S., et al., Nat. Commun. 2018, 9 (1), 2145 Elastin membranes were fabricated HDI crosslinking following our standard protocol, resulting in transparent membranes (FIG. 19A). As previously observed in ELP membranes32, elastin membranes also exhibited spherulitic structures throughout the membrane evidenced by both polarized microscopy (FIGS. 19A and 19B) and scanning electron microscopy (SEM) (FIGS. 19D and 19E).

Furthermore, elastin membranes were then exposed to a mineralization solution for 8 days as previously described using ELPs. Elsharkawy, S., et al., Nat. Commun. 2018, 9 (1), 2145; Deng, X., et al., Mater. Today Bio 2021, 11, 100119. Interestingly, elastin membranes exhibited a strong hierarchical mineralization emerging from the bulk of the membranes that was similar to that previously observed using ELPs (FIGS. 19D and 19E). In the results, such spherulitic structures were observed only within and on elastin membranes, following a similar structure and growth kinetics as those present in ELP membranes. These results demonstrate that elastin directs organized mineralization and acts as a platform for the formation of hierarchical mineral structures. These data suggest that elastin can be the point of calcification in soft tissues.

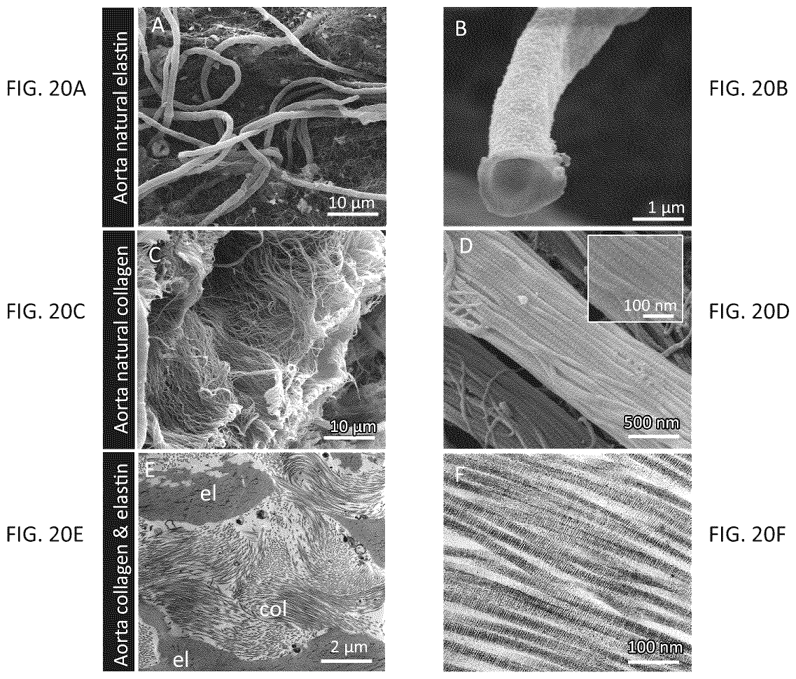

To test natural elastin mineralization's role in the onset of mineralization, investigations were conducted using bovine aortas and mitral valves. In the anatomy of the aorta, elastic lamella forms the basic unit of the tissue whereas collagen fibrils form the interlamellar matrix (FIG. 20). Here, elastic microfibrils are visible as entangled long filaments of about 1 μm diameter (FIGS. 20A and 20B). In the heart tissue, the organized collagen fibers are aligned parallel to the main axis of the smooth muscle cells, which each fibril formed by uniting each triple helix unit side by side into bundles (FIGS. 20C and 20D). Dingemans, K. P., et al., Anat. Rec. An Off. Publ. Am. Assoc. Anat. 2000, 258 (1), 1-14. Analyzing this further by TEM, it is possible to see that elastin regions are dense compared to collagen fibers that are organized in parallel to each other and at multiple orientations (FIGS. 20E and 20F). On the other hand, heart valves comprise natural elastin forming a core within elastic fibers encased by a microfibrillar sheath. Kodigepalli, K. M., et al., J. Cardiovasc. Dev. Dis. 2020, 7 (4), 57. Crosslinked elastin provides astonishing elasticity that endures deformation under small loads and can shrink back to the initial shape with minimum energy loss. The outer layer of valves that are in contact with outflow are formed by densely aligned collagen fibers that are providing the valves' strength. The distribution of both elastin and collagen proteins in heart tissue and their role in determining the mechanical properties is exigent. Therefore, elimination of each protein from the ECM of heart tissue, the changes in mineralization and mechanical features of the tissue can be studied in relation to each protein.

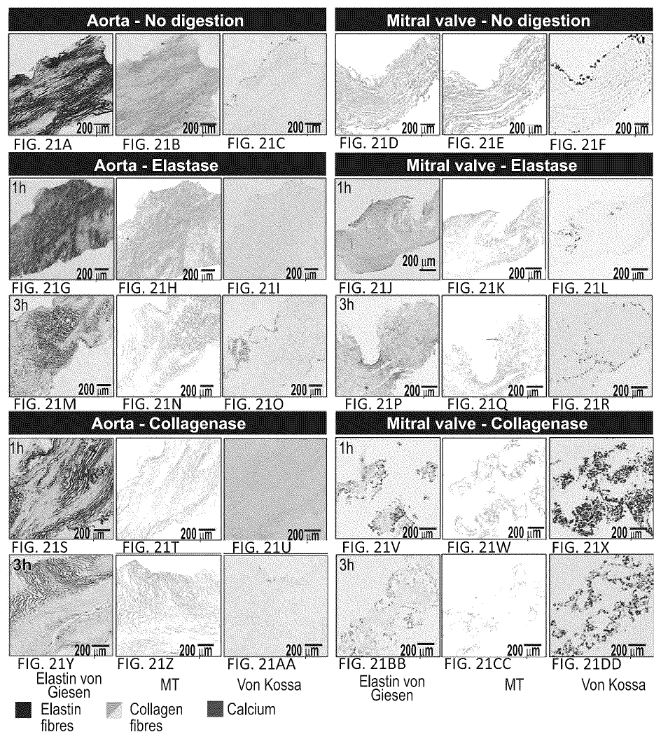

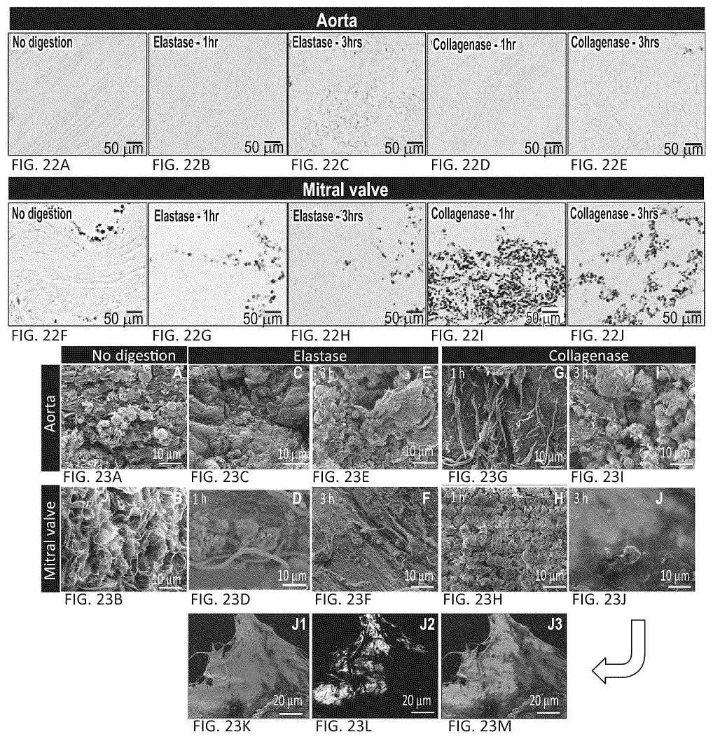

In this study, bovine aorta and mitral valve tissue samples were used to investigate the effects of both elastin and collagen in mineralization. To isolate the role of each protein, the tissues were digested with either elastase to generate collagen-rich tissues or collagenase to form elastin-rich tissues. The digestion parameters of concentration, incubation time, and temperature were adapted from published works by Fonck and Greenwald. Fonck, E., et al. Am. J. Physiol. Heart Circ. Physiol. 2007, 292 (6), H2754-63; Greenwald, S. E., et al., J. Biomech. Eng. 1997, 119 (4), 438-444. Elastin-stained (Elastin Von Giesen) histological sections revealed the presence of dark purple elastin filaments in aorta before digestion, which significantly decreased after digestion with elastase (FIGS. 21A-21DD). FIGS. 21A-21DD shows heart tissues stained for elastin using Elastin Von Giesen, collagen using Masson's trichome and calcium mineral using Von Kossa staining before digestion. Aorta and mitral valves were digested with Elastase and Collagenase for 1 hour and 3 hours then stained as other control tissues. In contrast, mitral valves displayed pale purple staining before digestion due to this tissue's inherently low content of elastin but is heavy in collagen content (FIGS. 21A-21DD). In the case of tissues digested with collagenase, the collagen content (blue/green color by Masson's Trichrome) decreased in aorta just after 1 hour of digestion. On the other hand, when the mitral valves were exposed to collagenase, significant amount of collagen were degraded. This resulted in drastic decrease in collagen content, where the mitral valve tissues lost its intactness (FIGS. 21A-21DD). At this stage of digestion, the elastin content started to be more visible in darker purple in mitral valve tissues (FIGS. 21A-21DD). Furthermore, as the mitral valve tissue is delicate and rich in collagen, after collagenase digestion, its consistency changed to a gel-like texture. Although, 3 hours of digestion was not enough to degrade all collagen and elastin content in both tissues. After the digestion of elastin and collagen contents, each tissue was incubated in mineralization solution for 8 days to further investigate the calcification ability in soft tissues.

Both aorta and mitral valve tissues were exposed to mineralization solution in order to assess their capacity to mineralize when a specific protein (either collagen or elastin) is isolated in an attempt to better investigate their role in generating Ca²+deposits (FIGS. 21A-21DD). These deposits were more evident in tissues digested by collagenase, where the elastin content of the ECM remains present (FIGS. 21A-21DD). Based on observation of Von Kossa staining of tissues, in both aorta and mitral valve, Ca²⁺ minerals formed along the elastin fibers (FIGS. 22A-22J showing higher magnification of tissues). Interestingly, Ca²⁺ was co-localized with elastin as confirmed by Von Kossa stain in tissues before and after digestion, specifically the samples that comprised higher proportions of elastin. (FIGS. 21A-21DD). These results confirm the strong mineralization potential of elastin and draw attention to its potential in the early stages of pathological calcification of heart tissues.

To further investigate the mineralizing role of elastin, all tissues were then analyzed via SEM observations. The results confirm the presence of mineral aggregates on the fibrillar structures of aorta and mitral valve before enzymatic digestion (FIGS. 23A and 23B). FIGS. 23A-23M display SEM images of aorta and mitral valve tissue before digestion (FIGS. 23A and 23B) showing minerals growing among the collagen fibers in aorta tissue. Elastin filaments are more evident in mitral valve tissue. Elastase digestion after 1 hour (FIGS. 23C and 23D) and 3 hours (FIGS. 23E and 23F) brings out the collagen fibers. Collagenase digestion after 1 hour (FIGS. 23G and 23H) and 3 hours (FIGS. 231 and 23J) showing heavily mineralized tissues. DDC-SEM and backscattered images of 3 hours-collagenase digested mitral valve (FIGS. 23K-23M) highlighting the dense mineralization, which further analyzed by backscattered electron microscopy. However, after digestions, the mineral growth was again more evident in tissues that were digested with collagenase and where elastin fibrils were more prevalent (FIGS. 23G-23J).

In contrast, tissues that were digested with Elastase exhibited mineralization alongside the collagen fibrils but at much lower quantities (FIGS. 23C-F). In collagenase digested tissues, densely mineralized structures were observed in both aorta and mitral valves specifically mitral valves that after just 1 hour of digestion with collagenase were covered with Ca²⁺ minerals (FIG. 23H) and after 3 hours of digestion the mineralization on the tissues was such that recreated the formations observed on the membrane-like structures (FIG. 23J) exhibiting spherulitic minerals (FIGS. 23K-23M). Interestingly, these kinds of spherulitic mineralized structures resemble those observed in human pathological cardiovascular tissues. Bertazzo, S., et al., Nat. Mater., 12(6): 576-583 (2013). To further investigate this, we conducted SEM observations on the mitral valves using the backscattered mode, which as expected revealed densely mineralized regions on the ECM of the tissues after 3-hour collagenase digestion. (orange area on DDC-SEM micrograph, FIG. 23M).

Table I below provides size measurements of elastin at varied CaCl₂ concentrations.

TABLE 1
Size measurements analysis of elastin
at different CaCl₂ concentrations.

| | | Elastin | | |
|---|---|---|---|---|
| | | Mean hydrodynamic | | |
| | | radius (nm) | Std. dev. | Mean PDI |
| 0 | mM CaCl₂ | 228.36 | 124.71 | 0.27 |
| 1 | mM CaCl₂ | 123.15 | 12.11 | 0.27 |
| 10 | mM CaCl₂ | 203.18 | 14.94 | 0.30 |
| 100 | mM CaCl₂ | 3059.42 | 2206.50 | 0.42 |

1. A polypeptide substrate comprising calcium ions, wherein the calcium ions are embedded within the polypeptide substrate.

2. A substrate according to claim 1 wherein the calcium ions are Ca²⁺.

3. A substrate according to claim 1, wherein the calcium ions are provided by CaCl₂.

4. A substrate according to claim 1, wherein the calcium ions are present in an amount of at least 0.001% by weight of the substrate.

5. A substrate according to claim 4, wherein the calcium ions are present in an amount of at least 0.005-1.5% by weight of the substrate.

6. A substrate according to claim 1, wherein the polypeptide is a pentapeptide Elastin-like-polyptide selected from the group consisting of Gly-X-X-X-X, X-Gly-X-X-X, X-X-Gly-X-X, X-X-X-Gly-X and X-X-X-X-Gly, (GXXXX, XGXXX, XXGXX, XXXGX, XXXXG), wherein X is any amino acid apart from proline.

7. A substrate according to claim 1, wherein the polypeptide is
MGSSHHHHHHSSGLVPRGSHMESLLP-
[((VPGIG)2VPGKG(VPGIG)2)2-
DDDEEKFLRRIGRFG-((VPGIG)2VPGKG(VPGIG)2)2]3-V.

8. A substrate according to claim 1, wherein the polypeptide substrate has a thickness of from 0.5 mm-1.5 mm.

9. A process for forming an elastin-like polypeptide membrane according to claim 1, the process comprising the steps of:
a) dissolving elastin-like polypeptides with a source of calcium ions and a solvent to form an ELP solution; and
b) applying the solution onto a surface to form a membrane.

10. A process according to claim 9, wherein the ELP is present in an amount of from 1-20% by weight of the solution.

11. A process according to claim 9, wherein the source of calcium ions may be present in an amount of 0.005-1.5% by volume of the solution.

12. A process according to claim 9, wherein the step a) further comprises the step of mixing the ELP solution with a cross-linker.

13. A process according to claim 12, wherein the cross-linker is hexamethyl diisocyanate.

14. A crystal formed from and at least partly embedded in a substrate according to claim 1.

15. A crystal according to claim 14, wherein the crystal is located at least partly inside the bulk of the polypeptide substrate.

16. A crystal according to claim 14, wherein the crystal is located partly inside the bulk of the polypeptide substrate and partly on the surface of the polypeptide substrate.

17. A crystal according to claim 14, wherein the crystal has a hierarchical structure.

18. A crystal according to claim 14, wherein the crystal comprises nanocrystals.

19. A crystal according to claim 18, wherein the nanocrystals are arranged in concentric layers.

20. A crystal according to claim 18, wherein the nanocrystals have a needle shape.

21. A crystal according to claim 20, wherein the needle shaped nanocrystals are located on the polypeptide substrate surface and orientated perpendicular to the polypeptide substrate surface.

22. A crystal according to claim 18, wherein the nanocrystals have a flower-liked shaped.

23. A crystal according to claim 18, wherein the nanocrystals within the substrate are fused.

24. A process for producing a crystal according to claim 14 comprising the steps of contacting a polypeptide substrate with a mineralizing solution.


SEQUENCE LISTING (43 sequences)

SEQ ID NO: 1 - protein, 4 aa, synthetic construct
  REGION 1..4: note = Bioactive epitope
RGDS

SEQ ID NO: 2 - protein, 15 aa, synthetic construct
  REGION 1..15: note = statherin-derived peptide
DDDEEKFLRRIGRFG

SEQ ID NO: 3-17 - skipped (15 entries)

SEQ ID NO: 18 - protein, 5 aa, synthetic construct
  REGION 1..5: note = Tropoelastin recurrent motif
  VARIANT 4: note = X is an amino acid selected from the group consisting of V, P, G, S, F and I
VPGXG

SEQ ID NO: 19 - protein, 5 aa, synthetic construct
  REGION 1..5: note = Tropoelastin recurrent motif
  VARIANT 4: note = X is any amino acid apart from Proline
VPGXG

SEQ ID NO: 20 - protein, 5 aa, synthetic construct
  REGION 1..5: note = Tropoelastin recurrent motif
  REGION 1..5: note = repeat_unit - Motif is to be repeated any number of times
  VARIANT 4: note = X is any amino acid apart from Proline
VPGXG

SEQ ID NO: 21 - protein, 5 aa, synthetic construct
  REGION 1..5: note = Tropoelastin recurrent motif
PGIPG

SEQ ID NO: 22 - protein, 5 aa, synthetic construct
  REGION 1..5: note = Tropoelastin recurrent motif
  REGION 1..5: note = repeat_unit - Motif is to be repeated any number of times
PGIPG

SEQ ID NO: 23 - protein, 5 aa, synthetic construct
  REGION 1..5: note = Tropoelastin recurrent motif
PVGSG

SEQ ID NO: 24 - protein, 5 aa, synthetic construct
  REGION 1..5: note = Tropoelastin recurrent motif
  REGION 1..5: note = repeat_unit - Motif is to be repeated any number of times
PVGSG

SEQ ID NO: 25 - protein, 5 aa, synthetic construct
  REGION 1..5: note = Tropoelastin recurrent motif
VGFPG

SEQ ID NO: 26 - protein, 5 aa, synthetic construct
  REGION 1..5: note = Tropoelastin recurrent motif
  REGION 1..5: note = repeat_unit - Motif is to be repeated any number of times
VGFPG

SEQ ID NO: 27 - protein, 699 aa, synthetic construct
  REGION 1..699: note = Peptide sequence
MGSSHHHHHHSSGLVPRGSHMESLLPVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGV

SEQ ID NO: 28 - protein, 877 aa, synthetic construct
  REGION 1..877: note = Peptide sequence
MESLLPVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGVPGIGVPGIGVPGKGVPGIGVPGIGEEIQIGHIPREDVDYHLYPVPGIGVPGIGVPGKGVPGIGVPGIGVGVAPGVGVAPGVGVAPGV

SEQ ID NO: 29 - protein, 1107 aa, synthetic construct
  REGION 1..1107: note = Peptide sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGV

SEQ ID NO: 30 - protein, 607 aa, synthetic construct
  REGION 1..607: note = Peptide sequence
MESLLPVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGV

SEQ ID NO: 31 - protein, 376 aa, synthetic construct
  REGION 1..376: note = Protein sequence
VPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGV

SEQ ID NO: 32 - protein, 352 aa, synthetic construct
  REGION 1..352: note = Peptide sequence
MESLLPVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGV

SEQ ID NO: 33 - protein, 452 aa, synthetic construct
  REGION 1..452: note = Peptide sequence
MESLLPVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGV

SEQ ID NO: 34 - protein, 797 aa, synthetic construct
  REGION 1..797: note = Peptide sequence
MESLLPVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGV

SEQ ID NO: 35 - protein, 457 aa, synthetic construct
  REGION 1..457: note = Peptide sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGV

SEQ ID NO: 36 - protein, 557 aa, synthetic construct
  REGION 1..557: note = Peptide sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGV

SEQ ID NO: 37 - protein, 707 aa, synthetic construct
  REGION 1..707: note = Peptide sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGV

SEQ ID NO: 38 - protein, 707 aa, synthetic construct
  REGION 1..707: note = Peptide sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPAVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGV

SEQ ID NO: 39 - protein, 698 aa, synthetic construct
  REGION 1..698: note = Bioactive sequence
MGSSHHHHHHSSGLVPRGSHMESLLPVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIG

SEQ ID NO: 40 - protein, 606 aa, synthetic construct
  REGION 1..606: note = ELP membrane sequence
MESLLPVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIG

SEQ ID NO: 41 - protein, 351 aa, synthetic construct
  REGION 1..351: note = ELP membrane sequence
MESLLPVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIG

SEQ ID NO: 42 - protein, 914 aa, synthetic construct
  REGION 1..914: note = ELP membrane sequence
MESLLPVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGDDDEEKFLRRIGRFGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIG

SEQ ID NO: 43 - protein, 5 aa, synthetic construct
  REGION 1..5: note = Tropoelastin recurrent motif
VPGIG